(19) United States
(12) Patent Application Publication
(10) Pub. No.: US 2026/0207558 A1
(43) Pub. Date: Jul. 23, 2026

(54) JAK1 PATHWAY INHIBITORS FOR THE TREATMENT OF VITILIGO

(71) Applicant: Incyte Corporation, Wilmington, DE (US)

(72) Inventors: Paul Smith, Carlsbad, CA (US); Kurt Andrew Brown, Narberth, PA (US); Michael D. Howell, Kennett Square, PA (US); Fiona Kuo, West Chester, PA (US); James Lee, Devon, PA (US); Leandro Luiz Dos Santos, King of Prussia, PA (US); Beth Rumberger, Quarryville, PA (US)

(21) Appl. No.: 19/569,964

(22) Filed: Mar. 17, 2026

Related U.S. Application Data
(63) Continuation of application No. 18/598,812, filed on Mar. 7, 2024.
Application No. 18/598,812 is a continuation of application No. 17/545,377, filed on Dec. 8, 2021, now Pat. No. 11,957,661.
(60) Provisional application No. 63/122,574, filed on Dec. 8, 2020.
(60) Provisional application No. 63/246,688, filed on Sep. 21, 2021.

(51) Int. Cl. A61K 31/4155; A61K 31/519; A61K 45/06; A61P 17/00

(52) U.S. Cl. CPC A61K 31/4155; A61K 31/519; A61K 45/06; A61P 17/00

(57) ABSTRACT

This disclosure relates to JAK1 pathway inhibitors and their use in treating vitiligo.

### PRIORITY CLAIM

This application is a continuation of U.S. application Ser. No. 17/545,377, filed on Dec. 8, 2021, which is claims the benefit of priority of U.S. Provisional Application Nos. 63/122,574, filed Dec. 8, 2020, and 63/246,688, filed Sep. 21, 2021, each of which is incorporated herein by reference in its entirety.

### TECHNICAL FIELD

This disclosure relates to JAK1 pathway inhibitors and the use thereof in treating vitiligo.

### BACKGROUND

Vitiligo is a chronic skin disorder characterized by depigmented patches of skin due to autoimmune destruction of melanocytes and is estimated to affect 0.5-2.0% of the global population (varying geographically). Vitiligo prevalence is similar between men and women, and there is no known difference in presentation based on skin type or race. Average age of onset is mid-twenties, but can occur at any age. The disorder tends to progress over time. 15-25% of people with vitiligo also tend to have at least one other autoimmune disorder. Non-segmental vitiligo is the most common subtype (up to 90% of vitiligo patients) and is characterized by symmetric bilateral white patches. The white patches are typically found on hands, feet and perioral/face.

There is a potential significant impact to quality of life for someone suffering from vitiligo due to psychosocial factors. The chronic nature of vitiligo and lack of effective therapy can have a negative psychosocial impact on patients, affecting quality of life similarly to other dermatological diseases. Involvement of cosmetically-sensitive areas, such as the face and hands, can have a major impact on self-esteem and eventually link to the psychological burden and quality of life; individuals with more than 25% of body surface area involvement may have difficulties performing daily tasks such as gardening, shopping, or clothing selection, and difficulty in socialization, like participating in sports activities and initiating and maintaining romantic relationships.

Clinical management aims to halt depigmentation and induce repigmentation. No drugs are currently approved for the treatment of patients with vitiligo. In general, first-line treatment consists of off-label use of topical corticosteroids and calcineurin inhibitors. Second line treatment consists of phototherapy (narrow-band UVB and psoralen and UVA) and systemic corticosteroids. Less common therapies include surgical grafting techniques and depigmenting treatments. Accordingly, there is a need to develop new therapies for the treatment of vitiligo. This application addresses this need and others.

### DESCRIPTION OF THE DRAWINGS

FIG. 1 is a graph depicting melanocyte proliferation, % confluence vs. time (hours).

FIG. 2 is a graph depicting the effect of Compound 1 on JAK1-dependent cytokines using T-cell compartments, protein T cell media (pg/ml) vs. specific JAK1-dependent cytokines.

FIGS. 3A-C are graphs depicting the effect of Compound 1 on growth factors and chemokine using T-cell compartments, protein T cell media (pg/ml) vs. IL-8 (A), HGF (B), and LIF (C).

FIG. 4 is a graph depicting the effect of Compound 1 on JAK1-dependent cytokines using melanocytes, protein melanocyte media (pg/ml) vs. specific JAK1-dependent cytokines.

FIGS. 5A-C are graphs depicting the effect of Compound 1 on growth factors and chemokine using melanocytes, protein melanocyte media (pg/ml) vs. IL-8 (A), HGF (B), and LIF (C).

FIG. 6 depicts an outline of a phase 2 randomized, double-blind, placebo-controlled dose-ranging study of the efficacy and safety of Compound 1.

FIG. 7 is a graph depicting melanocyte proliferation, % confluence vs. time (hours).

FIG. 8 is a graph depicting the effect of ruxolitinib on JAK1-dependent cytokines using T-cell compartments, protein T cell media (pg/ml) vs. specific JAK1-dependent cytokines.

FIGS. 9A-B are graphs depicting the effect of ruxolitinib on growth factors and chemokine using T-cell compartments, protein T cell media (pg/ml) vs. IL-8 (A) and LIF (B).

FIG. 10 is a graph depicting the effect of ruxolitinib on JAK1-dependent cytokines using melanocytes, protein melanocyte media (pg/ml) vs. specific JAK1-dependent cytokines.

FIGS. 11A-B are graphs depicting the effect of ruxolitinib on growth factors and chemokine using melanocytes, protein melanocyte media (pg/ml) vs. IL-8 (A) and LIF (B).

### SUMMARY

Provided herein are methods for the treatment of vitiligo in a subject in need thereof, comprising administering to the subject a therapeutically effective amount of a JAK1 pathway inhibitor, or a pharmaceutically acceptable salt thereof.

Provided herein is a JAK1 pathway inhibitor, or a pharmaceutically acceptable salt thereof, for the treatment of vitiligo in a subject in need thereof.

Provided herein is a use of a JAK1 pathway inhibitor, or a pharmaceutically acceptable salt thereof, for manufacture of a medicament for use in treating vitiligo in a subject in need thereof.

### DETAILED DESCRIPTION

The present invention provides, inter alia, a method of treating vitiligo in a subject in need thereof, comprising administering to the subject a therapeutically effective amount of a JAK1 pathway inhibitor, or a pharmaceutically acceptable salt thereof. In some embodiments, the vitiligo is non-segmental vitiligo.

In some embodiments, the present invention provides a method for treating vitiligo in a subject, said method comprising administering to the subject a therapeutically effective amount of a JAK1 pathway inhibitor, or a pharmaceutically acceptable salt thereof.

In some embodiments, the JAK1 pathway inhibitor, or a pharmaceutically acceptable salt thereof, is selective for JAK1 over JAK2, JAK3, and Tyk2.

In some embodiments, the JAK1 pathway inhibitor is 4-[3-(cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide, or a pharmaceutically acceptable salt thereof.

In some embodiments, the JAK1 pathway inhibitor is 4-[3-(cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide phosphoric acid salt.

In some embodiments, the vitiligo is non-segmental vitiligo.

In some embodiments, the JAK1 pathway inhibitor, or pharmaceutically acceptable salt thereof, is administered in a daily dose of about 5 mg to about 95 mg on a free base basis.

In some embodiments, the JAK1 pathway inhibitor, or pharmaceutically acceptable salt thereof, is administered in a daily dose of about 15 mg, about 45 mg, 75 mg, or about 90 mg on a free base basis.

In some embodiments, the JAK1 pathway inhibitor, or pharmaceutically acceptable salt thereof, is administered in combination with a further therapeutic agent.

In some embodiments, the further therapeutic agent comprises a Janus kinase inhibitor.

In some embodiments, the Janus kinase inhibitor comprises ruxolitinib, or a pharmaceutically acceptable salt thereof.

In some embodiments, the administering comprises administering the JAK1 pathway inhibitor, or pharmaceutically acceptable salt thereof, together with at least one pharmaceutically acceptable carrier or excipient.

In some embodiments, the vitiligo is non-segmental vitiligo. Non-segmental vitiligo is associated with some form of symmetry in the location of the patches of depigmentation. Classes of non-segmental vitiligo include generalized vitiligo, universal vitiligo, acrofacial vitiligo, mucosal vitiligo, and focal vitiligo. In some embodiments, the vitiligo is generalized vitiligo. In some embodiments, the vitiligo is universal vitiligo. In some embodiments, the vitiligo is acrofacial vitiligo. In some embodiments, the vitiligo is mucosal vitiligo. In some embodiments, the vitiligo is focal vitiligo. Generalized vitiligo, the most common category, affects approximately 0.5% of the world's population, with an average age of onset at about 24 years and occurring with approximately equal frequencies in males and females. While there is no variation by ethnicity, the disease can be much more apparent and thus emotionally distressing for individuals with darker skin colors.

In some embodiments, the vitiligo is segmental vitiligo. Segmental vitiligo differs in appearance, cause and prevalence than non-segmental vitiligo. Segmental vitiligo tends to affect areas of skin that are associated with dorsal roots from the spinal cord and is most often unilateral. Segmental vitiligo spreads much more rapidly than non-segmental vitiligo and, without treatment, segmental vitiligo is much more stable/static in course and is not associated with auto-immune diseases.

In some embodiments, efficacy of the treatment method disclosed herein can be established based upon percent change from various baseline measurements using various indicators. For example, total % of depigmented body surface area (BSA), including facial and nonfacial areas can be used. BSA can be assessed by the Palmar Method, as follows: the approximate size of the subject's entire palmar surface (ie, the palm plus 5 digits) should be considered 1% BSA, and the approximate size of the subject's thumb should be considered 0.1% BSA.

As used herein, the term “Face” may be generally defined as including the area on the forehead to the original hairline, on the cheek to the jawline vertically to the jawline and laterally from the corner of the mouth to the tragus. The area “Face” does not include surface area of the lips, scalp, ears, or neck but includes the nose and eyelids.

In some embodiments, a JAK1 inhibitor, e.g., Compound 1, and/or methods of use described herein result in about a 5%, about a 10%, about a 20%, about a 30%, about a 40%, about a 50%, about a 60%, about a 70%, about a 80%, about a 90%, or about a 95% reduction of depigmented Total Body Surface Area (T-BSA) in a subject.

In some embodiments, a JAK1 inhibitor, e.g., Compound 1, and/or methods of use described herein result in about a 5%, about a 10%, about a 20%, about a 30%, about a 40%, about a 50%, about a 60%, about a 70%, about a 80%, about a 90%, or about a 95% reduction of depigmented Facial Body Surface Area (F-BSA) in a subject.

In some embodiments, areas affected by depigmentation due to vitiligo can be assessed using the Vitiligo Area Scoring Index (VASI). VASI is based on a composite estimate of the percentage of vitiligo involvement (% BSA, using the Palmar Method) and the degree of depigmentation within the vitiligo lesions. In some embodiments, the degree of depigmentation can be determined and estimated to the nearest of the following percentages: 0%, 10%, 25%, 50%, 75%, 90%, or 100%. At 100% depigmentation, no pigment is present; at 90%, specks of pigment are present; at 75%, the depigmented area exceeds the pigmented area; at 50%, the depigmented and pigmented area are equal; at 25%, the pigmented area exceeds the depigmented area; at 10%, only specks of depigmentation are present. At 0% depigmentation, no depigmentation is present.

In some embodiments, the Facial Vitiligo Area Scoring Index (F-VASI) is then derived by multiplying the percentage of facial vitiligo involvement (% F-BSA) and the degree of depigmentation within the vitiligo lesions, and summing the values of all lesions together.

In some embodiments, efficacy may be measured using Total body VASI (T-VASI) which is derived by multiplying the percentage of total body vitiligo involvement (% T-BSA) and the degree of depigmentation within the vitiligo lesions for all body sites, and summing the values of all sites together (possible range, 0 to 100). For the purposes of T-VASI assessment, the body is divided into the following 6 separate and mutually exclusive sites: (1) head/neck (including scalp), (2) hands, (3) upper extremities (including axillae and excluding hands), (4) trunk (including genitalia, excluding buttocks), (5) lower extremities (including buttocks and excluding feet), and (6) feet.

In some embodiments, efficacy can be evaluated based upon percent change from baseline of T-VASI. In some embodiments, a JAK1 inhibitor, e.g., Compound 1, and/or methods of use described herein result in a reduction of T-VASI in a subject. In some embodiments, a JAK1 inhibitor, e.g., Compound 1, and/or methods of use described herein result in about a 50% or greater reduction of T-VASI in a subject. In some embodiments, a JAK1 inhibitor, e.g., Compound 1, and/or methods of use described herein result in about a 75% or greater reduction of T-VASI in a subject. In some embodiments, a JAK1 inhibitor, e.g., Compound 1, and/or methods of use described herein result in about a 90% or greater reduction of T-VASI in a subject. In some embodiments, a JAK1 inhibitor, e.g., Compound 1, and/or methods of use described herein result in about a 40% to about a 90% or greater reduction of T-VASI in a subject. In some embodiments, a JAK1 inhibitor, e.g., Compound 1, and/or methods of use described herein result in about a 50% to about a 95% or greater reduction of T-VASI in a subject. In some embodiments, a JAK1 inhibitor, e.g., Compound 1, and/or methods of use described herein result in about a 5%, about a 10%, about a 20%, about a 30%, about a 40%, about a 50%, about a 60%, about a 70%, about a 80%, about a 90%, or about a 95% reduction of T-VASI in a subject.

In some embodiments, efficacy can be evaluated based upon the proportion of subjects achieving about a 50% or greater reduction of T-VASI within a certain time period. In some embodiments, efficacy can be evaluated based upon the proportion of subjects achieving about a 50% or greater, about a 75% or greater, or about a 90% or greater reduction of T-VASI within a certain time period.

In some embodiments, efficacy can be evaluated based upon percent change from baseline of F-VASI. In some embodiments, a JAK1 inhibitor, e.g., Compound 1, and/or methods of use described herein result in a reduction of F-VASI in a subject. In some embodiments, a JAK1 inhibitor, e.g., Compound 1, and/or methods of use described herein result in about a 50% or greater reduction of F-VASI in a subject. In some embodiments, a JAK1 inhibitor, e.g., Compound 1, and/or methods of use described herein result in about a 75% or greater reduction of F-VASI in a subject. In some embodiments, a JAK1 inhibitor, e.g., Compound 1, and/or methods of use described herein result in about a 90% or greater reduction of F-VASI in a subject. In some embodiments, a JAK1 inhibitor, e.g., Compound 1, and/or methods of use described herein result in about a 40% to about a 90% or greater reduction of F-VASI in a subject. In some embodiments, a JAK1 inhibitor, e.g., Compound 1, and/or methods of use described herein result in about a 50% to about a 95% or greater reduction of F-VASI in a subject. In some embodiments, a JAK1 inhibitor, e.g., Compound 1, and/or methods of use described herein result in about a 5%, about a 10%, about a 20%, about a 30%, about a 40%, about a 50%, about a 60%, about a 70%, about a 80%, about a 90%, or about a 95% reduction of F-VASI in a subject.

The Vitiligo Extent Score (VES) can be a validated measure to express the overall vitiligo involvement of the body. The VES uses pictures of 19 separate anatomical areas for assessment of vitiligo extent (% BSA).

The Vitiligo Extent Score-plus (VESplus) can be a validated measure to express the overall vitiligo involvement of the body and a scale to assess the perifollicular repigmentation pattern. The VESplus uses pictures of 19 separate areas for assessment of vitiligo extent (% BSA) and their respective pattern of perifollicular repigmentation, divided in 7 categories: 0%, 5%, 10%, 25%, 50%, 75% and 90%.

In some embodiments, efficacy can be evaluated based upon improvement in the vitiligo extent and perifollicular repigmentation pattern using VESplus. Typically VESplus uses the calculation of the BSA times the degree of perifollicular repigmentation. In some embodiments, a JAK1 inhibitor, e.g., Compound 1, and/or methods of use described herein result in an improvement of VESplus for a subject. In some embodiments, a JAK1 inhibitor, e.g., Compound 1, and/or methods of use described herein result in a VESplus of about 50% or greater in a subject. In some embodiments, a JAK1 inhibitor, e.g., Compound 1, and/or methods of use described herein result in a VESplus of about 75% or greater in a subject. In some embodiments, a JAK1 inhibitor, e.g., Compound 1, and/or methods of use described herein result in a VESplus of about 90% or greater in a subject. In some embodiments, a JAK1 inhibitor, e.g., Compound 1, and/or methods of use described herein result in a VESplus of about 50% to about 90% or greater in a subject. In some embodiments, a JAK1 inhibitor, e.g., Compound 1, and/or methods of use described herein result in a VESplus of about 5% to about 75% or greater in a subject. In some embodiments, a JAK1 inhibitor, e.g., Compound 1, and/or methods of use described herein result in a VESplus of about 5% to about 90% or greater in a subject.

In some embodiments, efficacy can be evaluated based upon improvement in the Facial Static Investigator Global Assessment (FSIGA) score. The severity of facial vitiligo can be assessed using the FSIGA, which has a 5-point scale (Table 1).

TABLE 1

| Score | Severity | Description |
|---|---|---|
| 0 | Clear | No signs of vitiligo |
| 1 | Almost | Faint, barely detectable loss of pigmentation mainly |
| | Clear | located on forehead, periocular skin, lips, and/or |
| | | limited areas; approximately 90% pigmentation within |
| | | lesions; no or rare signs of Koebner phenomenon, |
| | | confetti-like or trichrome lesions may be present |
| 2 | Mild | Mild loss of pigmentation mainly located on |
| | Vitiligo | forehead, periocular skin, lips, and/or limited |
| | | areas; approximately 75% pigmentation within lesions; |
| | | few signs of Koebner phenomenon, confetti-like or |
| | | trichrome lesions may be present |
| 3 | Moderate | Moderate loss of pigmentation affecting several areas |
| | Vitiligo | of the face with large patches; approximately 50% |
| | | pigmentation within lesions; moderate number of signs |
| | | of Koebner phenomenon, confetti-like or trichrome |
| | | lesions may be present |
| 4 | Severe | Extensive loss of pigmentation affecting most areas |
| | Vitiligo | of the face; approximately 25% or less pigmentation |
| | | within lesions; many signs of Koebner phenomenon, |
| | | confetti-like or trichrome lesions affecting several |
| | | areas of the body may be present |

In some embodiments, a JAK1 inhibitor, e.g., Compound 1, and/or methods of use described herein result in a FSIGA score of clear (0) or almost clear (1) in a subject. In some embodiments, a JAK1 inhibitor, e.g., Compound 1, and/or methods of use described herein result in a FSIGA score improvement of at least 2 points. In some embodiments, a JAK1 inhibitor, e.g., Compound 1, and/or methods of use described herein result in a FSIGA score of clear (0) or almost clear (1) and at least a 2-point improvement in a subject.

In some embodiments, efficacy can be evaluated based upon improvement in the Static Investigator Global Assessment (SIGA) score. The severity of vitiligo can be assessed using the SIGA, which has a 5-point scale (Table 2).

TABLE 2

| Score | Severity | Description |
|---|---|---|
| 0 | Clear | No signs of vitiligo |
| 1 | Almost | Faint, barely detectable loss of pigmentation mainly |
| | Clear | located on dorsal hands, feet, bony prominences, |
| | | and/or limited areas; approximately 90% |
| | | pigmentation within lesions; no or rare signs of |
| | | Koebner phenomenon, confetti-like or trichrome |
| | | lesions may be present |
| 2 | Mild | Mild loss of pigmentation mainly located on |
| | Vitiligo | dorsal hands, feet, bony prominences, and/or |
| | | limited areas; approximately 75% pigmentation |
| | | within lesions; few signs of Koebner phenomenon, |
| | | confetti-like or trichrome lesions may be present |
| 3 | Moderate | Moderate loss of pigmentation affecting several |
| | Vitiligo | areas of the body with large patches; |
| | | approximately 50% pigmentation within lesions; |
| | | moderate number of signs of Koebner phenomenon, |
| | | confetti-like or trichrome lesions may be present |
| 4 | Severe | Extensive loss of pigmentation affecting most |
| | Vitiligo | areas of the body; approximately 25% or less |
| | | pigmentation within lesions; many signs of |
| | | Koebner phenomenon, confetti-like or trichrome |
| | | lesions affecting several areas of the body may |
| | | be present |

In some embodiments, a JAK1 inhibitor, e.g., Compound 1, and/or methods of use described herein result in a SIGA score of clear (0) or almost clear (1) in a subject. In some embodiments, a JAK1 inhibitor, e.g., Compound 1, and/or methods of use described herein result in a SIGA score improvement of at least 2 points. In some embodiments, a JAK1 inhibitor, e.g., Compound 1, and/or methods of use described herein result in a SIGA score improvement of at least 1, 2, or 3 points. In some embodiments, a JAK1 inhibitor, e.g., Compound 1, and/or methods of use described herein result in a SIGA score of clear (0) or almost clear (1) and at least a 2-point improvement in a subject.

Vitiligo is characterized by a progressive loss of functional melanocytes and associated with auditory abnormalities, including sensorineural hearing loss (SNHL) with a prevalence ranging from 4.0 to 68.8% when compared to healthy controls. During embryogenesis, precursor melanocyte cells (melanoblasts) migrate to the dermis, and also to the base of the cochlea, which is responsible for hearing high frequencies. It appears that inner ear melanocytes are very important for cochlear hair cell function and normal hearing. Persons suffering can undergo SNHL assessment at baseline to assess hearing, and throughout treatment to ascertain potential improvement in hearing relative to JAK1 inhibitor, e.g., Compound 1, exposure. In some embodiments, efficacy can be evaluated based upon improvement in a subject's hearing. In some embodiments, a JAK1 inhibitor, e.g., Compound 1, and/or methods of use described herein result in an improvement in a subject's baseline hearing parameters.

In some embodiments, a JAK1 inhibitor, e.g., Compound 1, and/or methods of use described herein result in about a 5%, about a 10%, about a 20%, about a 30%, about a 40%, about a 50%, about a 60%, about a 70%, about a 80%, about a 90%, or about a 95% improvement in a subject's baseline hearing parameters. In some embodiments, a JAK1 inhibitor, e.g., Compound 1, and/or methods of use described herein result in about a 50% or greater improvement in a subject's baseline hearing parameters.

In some embodiments, efficacy can be evaluated based upon improvement in a subject's response to a color-matching question (i.e., patient-reported outcomes (PROs)). In some embodiments, a JAK1 inhibitor, e.g., Compound 1, and/or methods of use described herein result in an improvement in a subject's response to a color-matching question. For the color-matching question the current subject' facial image (subjects may use a mirror) can be shown to the subject for reference, and the subject is asked to respond to the following query: “At this point of your treatment, how well does your skin color match between your face treated vitiligo skin and face normal skin?” Possible responses are: (1) Excellent, (2) Very good, (3) Good, (4) Poor, and (5) Very poor. In some embodiments, a JAK1 inhibitor, e.g., Compound 1, and/or methods of use described herein result in an improvement to the color-matching question of at least 2 points. In some embodiments, a JAK1 inhibitor, e.g., Compound 1, and/or methods of use described herein result in an improvement to the color-matching question of at least 1, 2, or 3 points.

In some embodiments, efficacy can be evaluated based upon improvement in a subject's assessed response, PRO, to a baseline facial photograph. In some embodiments, a JAK1 inhibitor, e.g., Compound 1, and/or methods of use described herein result in an improvement in a subject's assessed response to a baseline facial photograph. The Vitiligo Noticeability Scale (VNS) is a patient-reported measure of vitiligo treatment success, which has a 5-point scale. The baseline facial photograph can be shown to the subject for reference and a mirror can be provided for the subject to assess the vitiligo on their face. The subject can be asked to respond to the following query: “Compared with before treatment, how noticeable is the vitiligo now?” Possible responses are: (1) More noticeable, (2) As noticeable, (3) Slightly less noticeable, (4) A lot less noticeable, and (5) No longer noticeable. Compared with before treatment, how noticeable is the vitiligo now?Responses: (1) More noticeable, (2) As noticeable, (3) Slightly less noticeable, (4) A lot less noticeable, and (5) No longer noticeable. In some embodiments, a JAK1 inhibitor, e.g., Compound 1, and/or methods of use described herein result in a VNS score of 4 or 5.

In some embodiments, efficacy can be evaluated based upon improvement in a subject's response, PRO, to a Facial Patient Global Impression of Change-Vitiligo (F-PaGIC-V). In some embodiments, a JAK1 inhibitor, e.g., Compound 1, and/or methods of use described herein result in an improvement in a subject's response to F-PaGIC-V. The F-PaGIC-V is an assessment of improvement. It is a 7-point scale comparing the vitiligo areas at baseline with the subject's treated areas of facial vitiligo. The baseline photograph and current subjects' facial image (subject can be provided a mirror) will be shown to the subject for reference. The subject can be asked to respond to the following query: Since the start of the treatment you've received in this study, your vitiligo on your face treated with the study drug is: (1) Very much improved, (2) Much improved, (3) Minimally improved, (4) No change, (5) Minimally worse, (6) Much worse, and (7) Very much worse. In some embodiments, Compound 1 and/or methods of use described herein result in a F-PaGIC-V score of 1 or 2.

In some embodiments, efficacy can be evaluated based upon improvement in a subject's response, PRO, to a Total body Patient Global Impression of Change-Vitiligo (T-PaGIC-V). In some embodiments, a JAK1 inhibitor, e.g., Compound 1, and/or methods of use described herein result in an improvement in a subject's response to T-PaGIC-V. The T-PaGIC-V is an assessment of improvement. It is a 7-point scale comparing the vitiligo areas at baseline with the subject's treated areas of total body vitiligo. The subject can be asked to respond to the following query: Since the start of the treatment you've received in this study, your vitiligo on your total body treated with the study drug is: (1) Very much improved, (2) Much improved, (3) Minimally improved, (4) No change, (5) Minimally worse, (6) Much worse, and (7) Very much worse. In some embodiments, a JAK1 inhibitor, e.g., Compound 1, and/or methods of use described herein result in a T-PaGIC-V score of 1 or 2.

In some embodiments, efficacy can be evaluated based upon improvement, PRO, in a subject's Self-Assessment Vitiligo Extent Score (SA-VES). In some embodiments, a JAK1 inhibitor, e.g., Compound 1, and/or methods of use described herein result in an improvement in a subject's response to SA-VES from baseline. In some embodiments, a JAK1 inhibitor, e.g., Compound 1, and/or methods of use described herein result in about a 5%, about a 10%, about a 20%, about a 30%, about a 40%, about a 50%, about a 60%, about a 70%, about a 80%, about a 90%, or about a 95% improvement in a subject's response to SA-VES from baseline. The Self-Assessment Vitiligo Extent Score (SA-VES) can be a validated tool that allow patients to select the extent of the disease (presented via images) across 12 anatomical areas.

In some embodiments, efficacy can be evaluated based upon improvement, PRO, in a subject's Vitiligo Quality of Life (VitiQoL). In some embodiments, a JAK1 inhibitor, e.g., Compound 1, and/or methods of use described herein result in an improvement in a subject's response to VitiQoL from baseline. In some embodiments, a JAK1 inhibitor, e.g., Compound 1, and/or methods of use described herein result in about a 5%, about a 10%, about a 20%, about a 30%, about a 40%, about a 50%, about a 60%, about a 70%, about a 80%, about a 90%, or about a 95% improvement in a subject's response to VitiQoL from baseline. VitiQoL is a 15-item quality-of-life assessment that asks subjects to rate various aspects of their condition during the past month using a 7-point scale (“Not at all” to “All of the time”).

In some embodiments, efficacy can be evaluated based upon improvement, PRO, in a subject's Dermatology Life Quality Index (DLQI). In some embodiments, a JAK1 inhibitor, e.g., Compound 1, and/or methods of use described herein result in an improvement in a subject's response to DLQI from baseline. In some embodiments, a JAK1 inhibitor, e.g., Compound 1, and/or methods of use described herein result in about a 5%, about a 10%, about a 20%, about a 30%, about a 40%, about a 50%, about a 60%, about a 70%, about a 80%, about a 90%, or about a 95% improvement in a subject's response to DLQI from baseline. The DLQI is a 10-question validated questionnaire to measure how much the skin problem has affected the subject over the previous 7 days. Subjects can answer the questionnaire with (1) very much, (2) a lot, (3) a little, or (4) not at all. The questionnaire is analyzed under 6 headings as follows: Symptoms and feelings (Questions 1 and 2); Daily activities (Questions 3 and 4); Leisure (Questions 5 and 6); Work and school (Question 7); Personal relations (Questions 8 and 9); and Treatment (Question 10).

In some embodiments, efficacy can be evaluated based upon improvement, PRO, in a subject's Hospital Anxiety and Depression Scale (HADS). In some embodiments, a JAK1 inhibitor, e.g., Compound 1, and/or methods of use described herein result in an improvement in a subject's response to HADS from baseline. In some embodiments, a JAK1 inhibitor, e.g., Compound 1, and/or methods of use described herein result in about a 5%, about a 10%, about a 20%, about a 30%, about a 40%, about a 50%, about a 60%, about a 70%, about a 80%, about a 90%, or about a 95% improvement in a subject's response to HADS from baseline. HADS is 14-item questionnaire that assesses the levels of anxiety and depression that a person is currently experiencing. There are 7 questions each for measuring anxiety and for measuring depression, with 4 possible responses to each question (responses are scored as 0, 1, 2, or 3). Separate scores are calculated for anxiety and depression.

In some embodiments, efficacy can be evaluated based upon improvement, PRO, in a subject's WHO-5. In some embodiments, a JAK1 inhibitor, e.g., Compound 1, and/or methods of use described herein result in an improvement in a subject's response to WHO-5 from baseline. In some embodiments, a JAK1 inhibitor, e.g., Compound 1, and/or methods of use described herein result in about a 5%, about a 10%, about a 20%, about a 30%, about a 40%, about a 50%, about a 60%, about a 70%, about a 80%, about a 90%, or about a 95% improvement in a subject's response to WHO-5 from baseline. The WHO-5 can be a validated, self-administered, 5-item questionnaire designed to assess mental well-being over the past 2 weeks, which can be used as an outcome measure for the wanted and unwanted effects of treatments. The questionnaire consists of 5 statements, which respondents rate according to the following scale: 0=At no time; 1=Some of the time; 2=Less than half of the time; 3=More than half of the time; 4=Most of the time; 5=All of the time.

The raw score is calculated by totaling the figures of the 5 answers for a range of 0 to 25, with 0 representing the worst possible and 25 representing the best possible quality of life. A score below 13 indicates poor well-being.

In some embodiments, efficacy can be evaluated based upon improvement in a subject's Treatment Satisfaction Questionnaire for Medication (TSQM). In some embodiments, a JAK1 inhibitor, e.g., Compound 1, and/or methods of use described herein result in an improvement in a subject's response to TSQM from baseline. In some embodiments, a JAK1 inhibitor, e.g., Compound 1, and/or methods of use described herein result in about a 5%, about a 10%, about a 20%, about a 30%, about a 40%, about a 50%, about a 60%, about a 70%, about a 80%, about a 90%, or about a 95% improvement in a subject's response to TSQM from baseline. TSQM can be a validated 9-item questionnaire that measures a subject's satisfaction with treatment using a recall period of the past 2 to 3 weeks or since the treatment was last used. The questionnaire uses a 7-point scale for each question.

In some embodiments, efficacy can be evaluated based upon improvement in a subject's EQ-5D-5L questionnaire. In some embodiments, a JAK1 inhibitor, e.g., Compound 1, and/or methods of use described herein result in an improvement in a subject's response to the EQ-5D-5L questionnaire from baseline. In some embodiments, a JAK1 inhibitor, e.g., Compound 1, and/or methods of use described herein result in about a 5%, about a 10%, about a 20%, about a 30%, about a 40%, about a 50%, about a 60%, about a 70%, about a 80%, about a 90%, or about a 95% improvement in a subject's response to EQ-5D-5L from baseline. The EQ-5D-5L questionnaire is a standardized, validated instrument for use as a measure of health outcome. The EQ-5D-5L questionnaire will provide data for use in economic models and analyses, including developing health utilities or Quality-Adjusted Life Years (QALYs). The EQ-5D-5L questionnaire consists of the following 2 sections: the EQ-5D descriptive system and the EQ VAS. The descriptive system comprises five dimensions: mobility, self-care, usual activities, pain/discomfort, and anxiety/depression.

Each dimension has 5 levels: Level 1 is “no problems,” Level 2 is “slight problems,” Level 3 is “moderate problems,” Level 4 is “severe problems,” and Level 5 is “extreme problems.” This part of the EQ-5D-5L questionnaire provides a descriptive profile that can be used to generate a health state profile. For example, a subject in “health state 12345” would have no problems with mobility, slight problems with self-care (washing or dressing), moderate problems with doing usual activities, severe pain or discomfort and extreme anxiety or depression. Each health state can potentially be assigned a summary index score based on societal preference weights for the health state. These weights, sometimes referred to as utilities, are often used to compute QALYs for use in health economic analyses. Health state index scores generally range from less than 0 (where 0 is the value of a health state equivalent to dead; negative values representing values as worse than dead) to 1 (the value of full health), with higher scores indicating higher health utility. The health state preferences often represent national or regional values and can therefore differ between countries/regions. The EQ VAS records the subject's self-rated health on a vertical visual analogue scale (0 to 100), where the endpoints are labelled “the best health you can imagine” (100 score) and “the worst health you can imagine” (0 score).

The methods described herein utilize JAK1 pathway inhibitors, particularly JAK1 selective inhibitors. A JAK1 selective inhibitor is a compound that inhibits JAK1 activity preferentially over other Janus kinases. JAK1 plays a central role in a number of cytokine and growth factor signaling pathways that, when dysregulated, can result in or contribute to disease states. For example, levels of IL-6 (a pro-inflammatory cytokine) are elevated in subjects suffering from vitiligo (Singh, et al., Indian J Dermatology, 2012 January-February; 57(1): 12-14). In other autoimmune diseases and cancers, elevated systemic levels of inflammatory cytokines that activate JAK1 may also contribute to the disease and/or associated symptoms. Therefore, patients with autoimmune diseases like vitiligo may benefit from JAK1 inhibition. Selective inhibitors of JAK1 may be efficacious while avoiding unnecessary and potentially undesirable effects of inhibiting other JAK kinases.

In some embodiments, the JAK1 pathway inhibitor, or a pharmaceutically acceptable salt thereof, is selective for JAK1 over JAK2, JAK3, and TYK2 (i.e., a JAK1 selective inhibitor). For example, the compounds described herein, or pharmaceutically acceptable salts thereof, preferentially inhibit JAK1 over one or more of JAK2, JAK3, and TYK2. In some embodiments, the compounds inhibit JAK1 preferentially over JAK2 (e.g., have a JAK2/JAK1 IC₅₀ ratio >1). In some embodiments, the compounds or salts are about 10-fold more selective for JAK1 over JAK2. In some embodiments, the compounds or salts are about 3-fold, about 5-fold, about 10-fold, about 15-fold, or about 20-fold more selective for JAK1 over JAK2 as calculated by measuring IC₅₀ at 1 mM ATP (e.g., see Example A).

In some embodiments, the JAK1 pathway inhibitor is a compound of Table 3, or a pharmaceutically acceptable salt thereof. The compounds in Table 3 are selective JAK1 inhibitors (selective over JAK2, JAK3, and TYK2). The IC₅₀ values obtained by the method of Example A at 1 mM ATP are shown in Table 3.

The compounds of Table 3 can be prepared by the synthetic procedures described, for example, in US Patent Publ. No. 2011/0224190, filed Mar. 9, 2011, US Patent Publ. No. 2014/0343030, filed May 16, 2014, US Patent Publ. No. 2014/0121198, filed Oct. 31, 2013, US Patent Publ. No. 2010/0298334, filed May 21, 2010, US Patent Publ. No. 2011/0059951, filed Aug. 31, 2010, US Patent Publ. No. 2012/0149681, filed Nov. 18, 2011, US Patent Publ. No. 2012/0149682, filed Nov. 18, 2011, US Patent Publ. 2013/0018034, filed Jun. 19, 2012, US Patent Publ. No. 2013/0045963, filed Aug. 17, 2012, and US Patent Publ. No. 2014/0005166, filed May 17, 2013, each of which is incorporated herein by reference in its entirety.

TABLE 3

| | | | | JAK1 | |
|---|---|---|---|---|---|
| Comp. | | | | IC₅₀ | JAK2/ |
| No. | Prep. | Name | Structure | (nM) | JAK1 |
| 1 | US 2014/ 0343030 (Example 7) | 4-[3-(cyanomethyl)-3-(3′,5′- dimethyl-1H,1′H-4,4′- bipyrazol-1-yl)azetidin-1- yl]-2,5-difluoro-N-[(1S)- 2,2,2-trifluoro-1- methylethyl]benzamide | 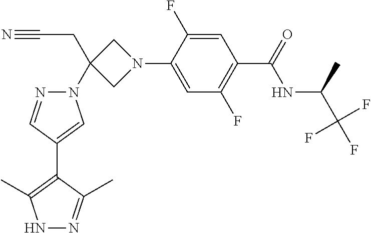 [SMILES: Cc1n[nH]c(C)c1-c1cnn(C2(CC#N)CN(c3cc(F)c(C(=O)N[C@@H](C)C(F)(F)F)cc3F)C2)c1] | +++ | >10 |
| 2 | US 2011/ 0224190 (Example 154) | 4-{3-(Cyanomethyl)-3-[4- (7H-pyrrolo[2,3- d]pyrimidin-4-yl)-1H- pyrazol-1-yl]azetidin-1-yl}- N-[4-fluoro-2- (trifluoromethyl)phenyl] piperidine-1-carboxamide | 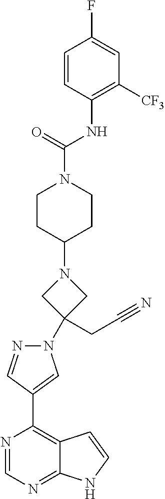 [SMILES: N#CCC1(n2cc(-c3ncnc4[nH]ccc34)cn2)CN(C2CCN(C(=O)Nc3ccc(F)cc3C(F)(F)F)CC2)C1] | + | >10 |
| 3 | US 2011/ 0224190 (Example 85) | [3-[4-(7H-pyrrolo[2,3- d]pyrimidin-4-yl)-1H- pyrazol-1-yl]-1-(1-{[2- (trifluoromethyl)pyrimidin- 4-yl]carbonyl}piperidin-4- yl)azetidin-3-yl]acetonitrile | 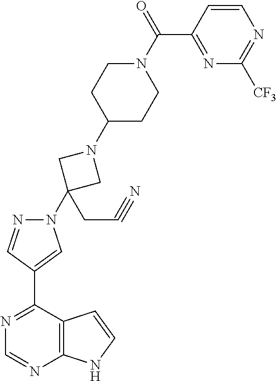 [SMILES: N#CCC1(n2cc(-c3ncnc4[nH]ccc34)cn2)CN(C2CCN(C(=O)c3ccnc(C(F)(F)F)n3)CC2)C1] | + | >10 |
| 4 | US 2011/ 0224190 (Example 1) | {1-{1-[3-Fluoro-2- (trifluoromethyl) isonicotinoyl] piperidin-4-yl}-3-[4- (7H-pyrrolo[2,3- d]pyrimidin-4-yl)-1H- pyrazol-1-yl]azetidin-3- yl}acetonitrile | 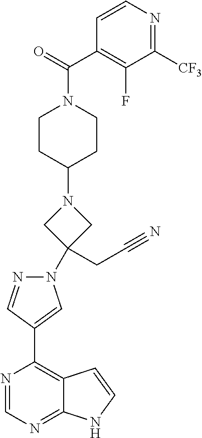 [SMILES: N#CCC1(n2cc(-c3ncnc4[nH]ccc34)cn2)CN(C2CCN(C(=O)c3ccnc(C(F)(F)F)c3F)CC2)C1] | + | >10 |
| 5 | US 2014/ 0121198 (Example 20) | ((2R,5S)-5-{2-[(1R)-1- hydroxyethyl]-1H- imidazo[4,5-d]thieno[3,2- b]pyridin-1-yl}tetrahydro- 2H-pyran-2-yl)acetonitrile | 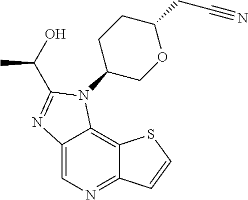 [SMILES: C[C@@H](O)c1nc2cnc3ccsc3c2n1[C@H]1CC[C@H](CC#N)OC1] | ++ | >10 |
| 6 | US 2010/ 0298334 (Example 2)^{a} | 3-[1-(6-chloropyridin-2- yl)pyrrolidin-3-yl]-3-[4- (7H-pyrrolo[2,3- d]pyrimidin-4-yl)-1H- pyrazol-1-yl] propanenitrile | 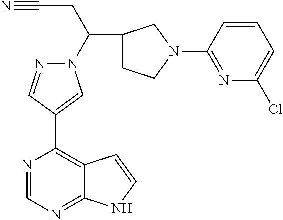 [SMILES: N#CCC(C1CCN(c2cccc(Cl)n2)C1)n1cc(-c2ncnc3[nH]ccc23)cn1] | + | >10 |
| 7 | US 2010/ 0298334 (Example 13c) | 3-(1-[1,3]oxazolo[5,4- b]pyridin-2-ylpyrrolidin- 3-yl)-3-[4-(7H-pyrrolo [2,3-d]pyrimidin-4-yl)- 1H-pyrazol-1-yl] propanenitrile | 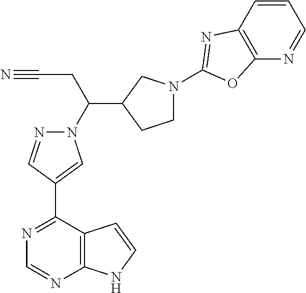 [SMILES: N#CCC(C1CCN(c2nc3cccnc3o2)C1)n1cc(-c2ncnc3[nH]ccc23)cn1] | + | >10 |
| 8 | US 2011/ 0059951 (Example 12) | 4-[(4-{3-cyano-2-[4-(7H- pyrrolo[2,3-d]pyrimidin- 4-yl)-1H-pyrazol-1- yl]propyl}piperazin-1- yl)carbonyl]-3- fluorobenzonitrile | 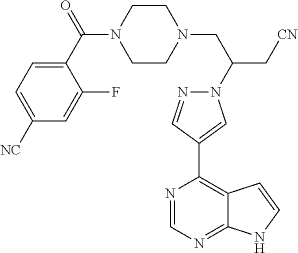 [SMILES: N#CCC(CN1CCN(C(=O)c2ccc(C#N)cc2F)CC1)n1cc(-c2ncnc3[nH]ccc23)cn1] | + | >10 |
| 9 | US 2011/ 0059951 (Example 13) | 4-[(4-{3-cyano-2-[3-(7H- pyrrolo[2,3-d]pyrimidin- 4-yl)-1H-pyrrol-1- yl]propyl}piperazin-1- yl)carbonyl]-3- fluorobenzonitrile | 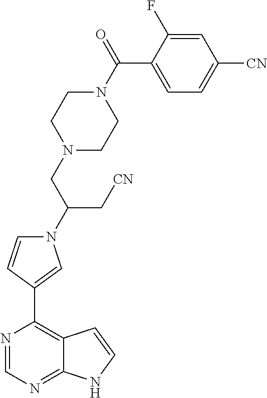 [SMILES: N#CCC(CN1CCN(C(=O)c2ccc(C#N)cc2F)CC1)n1ccc(-c2ncnc3[nH]ccc23)c1] | + | >10 |
| 10 | US 2012/ 0149681 (Example 7b) | [trans-1-[4-(7H- pyrrolo[2,3-d]pyrimidin-4- yl)-1H-pyrazol-1-yl]-3-(4- {[2- (trifluoromethyl)pyrimidin- 4-yl]carbonyl}piperazin-1- yl)cyclobutyl]acetonitrile | 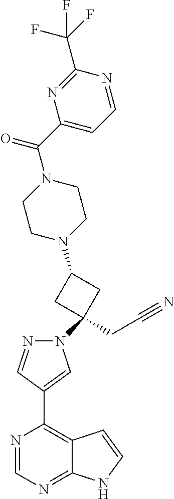 [SMILES: N#CC[C@]1(n2cc(-c3ncnc4[nH]ccc34)cn2)C[C@H](N2CCN(C(=O)c3ccnc(C(F)(F)F)n3)CC2)C1] | + | >10 |
| 11 | US 2012/ 0149681 (Example 157) | {trans-3-(4-{[4-[(3- hydroxyazetidin-1- yl)methyl]-6- (trifluoromethyl)pyridin-2- yl]oxy}piperidin-1-yl)-1- [4-(7H-pyrrolo[2,3- d]pyrimidin-4-yl)-1H- pyrazol-1- yl]cyclobutyl}acetonitrile | 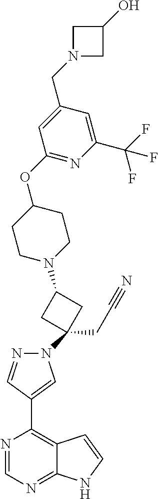 [SMILES: N#CC[C@]1(n2cc(-c3ncnc4[nH]ccc34)cn2)C[C@H](N2CCC(Oc3cc(CN4CC(O)C4)cc(C(F)(F)F)n3)CC2)C1] | + | >10 |
| 12 | US 2012/ 0149681 (Example 161) | {trans-3-(4-{[4-{[(2S)-2- (hydroxymethyl)pyrrolidin- 1-yl]methyl}-6- (trifluoromethyl)pyridin-2- yl]oxy}piperidin-1-yl)-1- [4-(7H-pyrrolo[2,3- d]pyrimidin-4-yl)-1H- pyrazol-1- yl]cyclobutyl}acetonitrile | 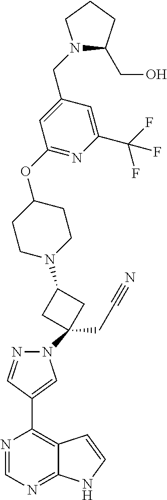 [SMILES: N#CC[C@]1(n2cc(-c3ncnc4[nH]ccc34)cn2)C[C@H](N2CCC(Oc3cc(CN4CCC[C@H]4CO)cc(C(F)(F)F)n3)CC2)C1] | + | >10 |
| 13 | US 2012/ 0149681 (Example 162) | {trans-3-(4-{[4-{[(2R)-2- (hydroxymethyl)pyrrolidin- 1-yl]methyl}-6- (trifluoromethyl)pyridin-2- yl]oxy}piperidin-1-yl)-1- [4-(7H-pyrrolo[2,3- d]pyrimidin-4-yl)-1H- pyrazol-1- yl]cyclobutyl}acetonitrile | 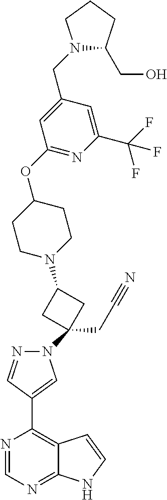 [SMILES: N#CC[C@]1(n2cc(-c3ncnc4[nH]ccc34)cn2)C[C@H](N2CCC(Oc3cc(CN4CCC[C@@H]4CO)cc(C(F)(F)F)n3)CC2)C1] | + | >10 |
| 14 | US 2012/ 0149682 (Example 20)^{b} | 4-(4-{3- [(dimethylamino)methyl]- 5-fluorophenoxy}piperidin- 1-yl)-3-[4-(7H-pyrrolo[2,3- d]pyrimidin-4-yl)-1H- pyrazol-1-yl]butanenitrile | 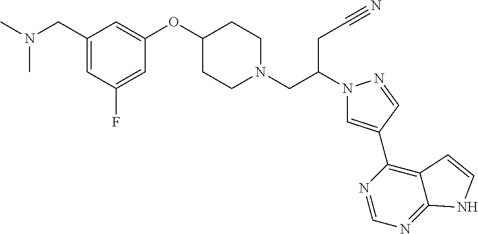 [SMILES: CN(C)Cc1cc(F)cc(OC2CCN(CC(CC#N)n3cc(-c4ncnc5[nH]ccc45)cn3)CC2)c1] | + | >10 |
| 15 | US 2013/ 0018034 (Example 18) | 5-{3-(cyanomethyl)-3-[4- (7H-pyrrolo[2,3- d]pyrimidin-4-yl)-1H- pyrazol-1-yl]azetidin-1-yl}- N-isopropylpyrazine-2- carboxamide | 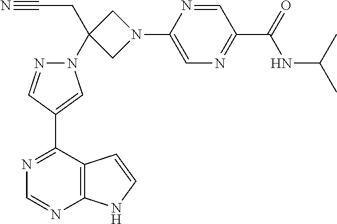 [SMILES: CC(C)NC(=O)c1cnc(N2CC(CC#N)(n3cc(-c4ncnc5[nH]ccc45)cn3)C2)cn1] | + | >10 |
| 16 | US 2013/ 0018034 (Example 28) | 4-{3-(cyanomethyl)-3-[4- (7H-pyrrolo[2,3- d]pyrimidin-4-yl)-1H- pyrazol-1-yl]azetidin-1-yl}- 2,5-difluoro-N-[(1S)-2,2,2- trifluoro-1- methylethyl]benzamide | 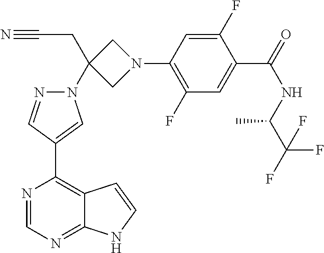 [SMILES: C[C@H](NC(=O)c1cc(F)c(N2CC(CC#N)(n3cc(-c4ncnc5[nH]ccc45)cn3)C2)cc1F)C(F)(F)F] | + | >10 |
| 17 | US 2013/ 0018034 (Example 34) | 5-{3-(cyanomethyl)-3-[4- (1H-pyrrolo[2,3-b]pyridin- 4-yl)-1H-pyrazol-1- yl]azetidin-1-yl}-N- isopropylpyrazine-2- carboxamide | 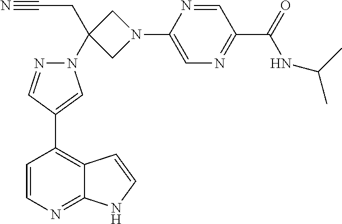 [SMILES: CC(C)NC(=O)c1cnc(N2CC(CC#N)(n3cc(-c4ccnc5[nH]ccc45)cn3)C2)cn1] | + | >10 |
| 18 | US 2013/ 0045963 (Example 45) | {1-(cis-4-{[6-(2- hydroxyethyl)-2- (trifluoromethyl)pyrimidin- 4-yl]oxy}cyclohexyl)-3-[4- (7H-pyrrolo[2,3- d]pyrimidin-4-yl)-1H- pyrazol-1-yl]azetidin-3- yl}acetonitrile | 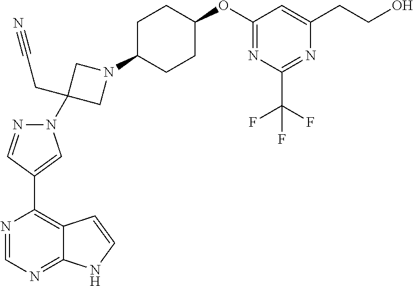 [SMILES: N#CCC1(n2cc(-c3ncnc4[nH]ccc34)cn2)CN([C@H]2CC[C@@H](Oc3cc(CCO)nc(C(F)(F)F)n3)CC2)C1] | + | >10 |
| 19 | US 2013/ 0045963 (Example 65) | {1-(cis-4-{[4- [(ethylamino)methyl]-6- (trifluoromethyl)pyridin-2- yl]oxy}cyclohexyl)-3-[4- (7H-pyrrolo[2,3- d]pyrimidin-4-yl)-1H- pyrazol-1-yl]azetidin-3- yl}acetonitrile | 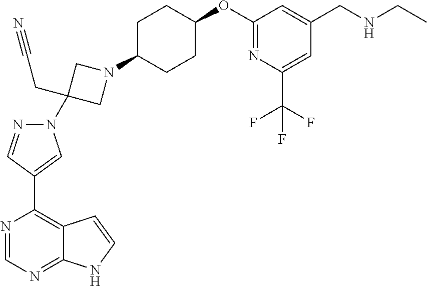 [SMILES: CCNCc1cc(O[C@H]2CC[C@@H](N3CC(CC#N)(n4cc(-c5ncnc6[nH]ccc56)cn4)C3)CC2)nc(C(F)(F)F)c1] | + | >10 |
| 20 | US 2013/ 0045963 (Example 69) | {1-(cis-4-{[4-(1-hydroxy-1- methylethyl)-6- (trifluoromethyl)pyridin-2- yl]oxy}cyclohexyl)-3-[4- (7H-pyrrolo[2,3- d]pyrimidin-4-yl)-1H- pyrazol-1-yl]azetidin-3- yl}acetonitrile | 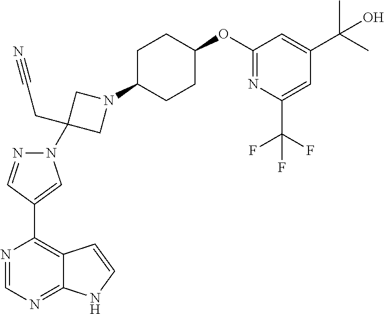 [SMILES: CC(C)(O)c1cc(O[C@H]2CC[C@@H](N3CC(CC#N)(n4cc(-c5ncnc6[nH]ccc56)cn4)C3)CC2)nc(C(F)(F)F)c1] | + | >10 |
| 21 | US 2013/ 0045963 (Example 95) | {1-(cis-4-{[4-{[(3R)-3- hydroxypyrrolidin-1- yl]methyl}-6- (trifluoromethyl)pyridin-2- yl]oxy}cyclohexyl)-3-[4- (7H-pyrrolo[2,3- d]pyrimidin-4-yl)-1H- pyrazol-1-yl]azetidin-3- yl}acetonitrile | 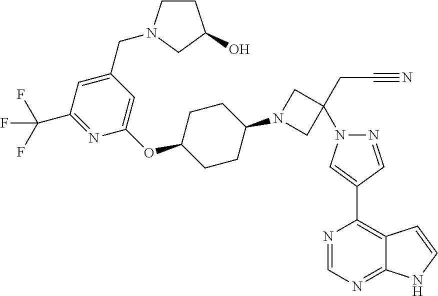 [SMILES: N#CCC1(n2cc(-c3ncnc4[nH]ccc34)cn2)CN([C@H]2CC[C@@H](Oc3cc(CN4CC[C@@H](O)C4)cc(C(F)(F)F)n3)CC2)C1] | + | >10 |
| 22 | US 2013/ 0045963 (Example 95) | {1-(cis-4-{[4-{[(3S)-3- hydroxypyrrolidin-1- yl]methyl}-6- (trifluoromethyl)pyridin-2- yl]oxy}cyclohexyl)-3-[4- (7H-pyrrolo[2,3- d]pyrimidin-4-yl)-1H- pyrazol-1-yl]azetidin-3- yl}acetonitrile | 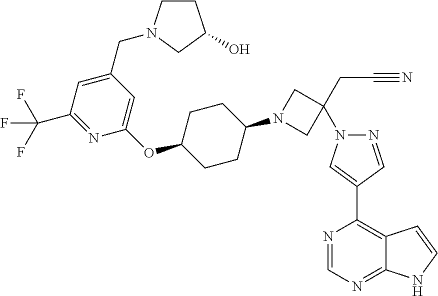 [SMILES: N#CCC1(n2cc(-c3ncnc4[nH]ccc34)cn2)CN([C@H]2CC[C@@H](Oc3cc(CN4CC[C@H](O)C4)cc(C(F)(F)F)n3)CC2)C1] | + | >10 |
| 23 | US 2014/ 0005166 (Example 1) | {trans-3-(4-{[4-({[(1S)-2- hydroxy-1- methylethyl]amino}methyl)- 6-(trifluoromethyl)pyridin- 2-yl]oxy}piperidin-1-yl)-1- [4-(7H-pyrrolo[2,3- d]pyrimidin-4-yl)-1H- pyrazol-1- yl]cyclobutyl}acetonitrile | 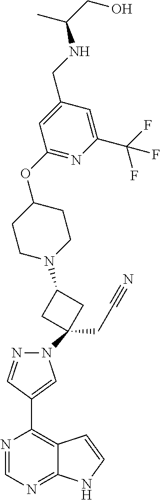 [SMILES: C[C@@H](CO)NCc1cc(OC2CCN([C@H]3C[C@](CC#N)(n4cc(-c5ncnc6[nH]ccc56)cn4)C3)CC2)nc(C(F)(F)F)c1] | + | >10 |
| 24 | US 2014/ 0005166 (Example 14) | {trans-3-(4-{[4-({[(2R)-2- hydroxypropyl]amino}meth- yl)-6- (trifluoromethyl)pyridin-2- yl]oxy}piperidin-1-yl)-1- [4-(7H-pyrrolo[2,3- d]pyrimidin-4-yl)-1H- pyrazol-1- yl]cyclobutyl}acetonitrile | 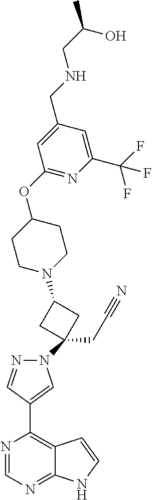 [SMILES: C[C@@H](O)CNCc1cc(OC2CCN([C@H]3C[C@](CC#N)(n4cc(-c5ncnc6[nH]ccc56)cn4)C3)CC2)nc(C(F)(F)F)c1] | + | >10 |
| 25 | US 2014/ 0005166 (Example 15) | {trans-3-(4-{[4-({[(2S)-2- hydroxypropyl]amino}meth- yl)-6- (trifluoromethyl)pyridin-2- yl]oxy}piperidin-1-yl)-1- [4-(7H-pyrrolo[2,3- d]pyrimidin-4-yl)-1H- pyrazol-1- yl]cyclobutyl}acetonitrile | 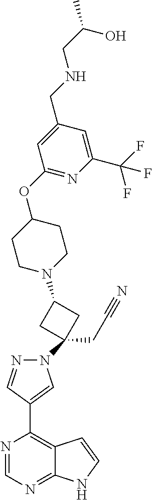 [SMILES: C[C@H](O)CNCc1cc(OC2CCN([C@H]3C[C@](CC#N)(n4cc(-c5ncnc6[nH]ccc56)cn4)C3)CC2)nc(C(F)(F)F)c1] | + | >10 |
| 26 | US 2014/ 0005166 (Example 20) | {trans-3-(4-{[4-(2- hydroxyethyl)-6- (trifluoromethyl)pyridin-2- yl]oxy}piperidin-1-yl)-1- [4-(7H-pyrrolo[2,3- d]pyrimidin-4-yl)-1H- pyrazol-1- yl]cyclobutyl}acetonitrile | 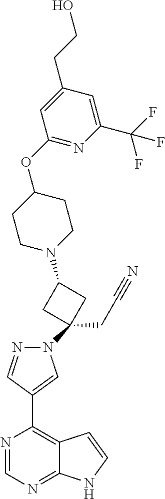 [SMILES: N#CC[C@]1(n2cc(-c3ncnc4[nH]ccc34)cn2)C[C@H](N2CCC(Oc3cc(CCO)cc(C(F)(F)F)n3)CC2)C1] | + | >10 |

+ means <10 nM (see Example A for assay conditions)
++ means ≤100 nM (see Example A for assay conditions)
+++ means ≤300 nM (see Example A for assay conditions)
^{a}Data for enantiomer 1
^{b}Data for enantiomer 2

In some embodiments, the JAK1 pathway inhibitor is 4-[3-(cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide (Compound 1), or a pharmaceutically acceptable salt thereof. In some embodiments, the JAK1 pathway inhibitor is 4-[3-(cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide phosphoric acid salt. Compound 1, and its salts, can be made by the procedures described in, e.g., U.S. Pat. No. 9,382,231 (see, e.g., Example 7), filed May 16, 2014, which is incorporated herein by reference in its entirety.

In some embodiments, the JAK1 pathway inhibitor is selected from the compounds, or pharmaceutically acceptable salts thereof, described in US Patent Publ. No. 2011/0224190, filed Mar. 9, 2011, US Patent Publ. No. 2014/0343030, filed May 16, 2014, US Patent Publ. No. 2014/0121198, filed Oct. 31, 2013, US Patent Publ. No. 2010/0298334, filed May 21, 2010, US Patent Publ. No. 2011/0059951, filed Aug. 31, 2010, US Patent Publ. No. 2012/0149681, filed Nov. 18, 2011, US Patent Publ. No. 2012/0149682, filed Nov. 18, 2011, US Patent Publ. 2013/0018034, filed Jun. 19, 2012, US Patent Publ. No. 2013/0045963, filed Aug. 17, 2012, and US Patent Publ. No. 2014/0005166, filed May 17, 2013, each of which is incorporated herein by reference in its entirety.

In some embodiments, the JAK1 pathway inhibitor is a compound of Formula I

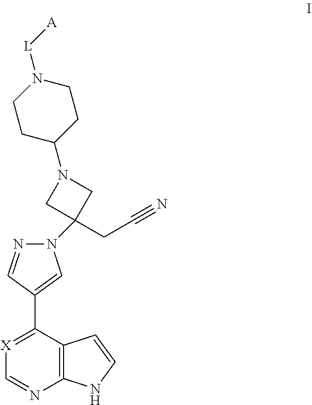

or a pharmaceutically acceptable salt thereof, wherein:
X is N or CH;
L is C(═O) or C(═O)NH;
A is phenyl, pyridinyl, or pyrimidinyl each of which is optionally substituted with 1 or 2 independently selected R¹ groups; and
each R¹ is, independently, fluoro, or trifluoromethyl.

In some embodiments, the compound of Formula I is {1-{1-[3-fluoro-2-(trifluoromethyl)isonicotinoyl]piperidin-4-yl}-3[4-(7H-pyrrolo[2,3-d]pyrimidin-4-yl)-1H-pyrazol-1-yl]azetidin-3-yl}acetonitrile, or a pharmaceutically acceptable salt thereof.

In some embodiments, the compound of Formula I is 4-{3-(Cyanomethyl)-3-[4-(7H-pyrrolo[2,3-d]pyrimidin-4-yl)-1H-pyrazol-1-yl]azetidin-1-yl}-N-[4-fluoro-2-(trifluoromethyl)phenyl]piperidine-1-carboxamide, or a pharmaceutically acceptable salt thereof.

In some embodiments, the compound of Formula I is [3-[4-(7H-pyrrolo[2,3-d]pyrimidin-4-yl)-1H-pyrazol-1-yl]-1-(1-{[2-(trifluoromethyl)pyrimidin-4-yl]carbonyl}piperidin-4-yl)azetidin-3-yl]acetonitrile, or a pharmaceutically acceptable salt thereof.

In some embodiments, the JAK1 pathway inhibitor is a compound of Formula II

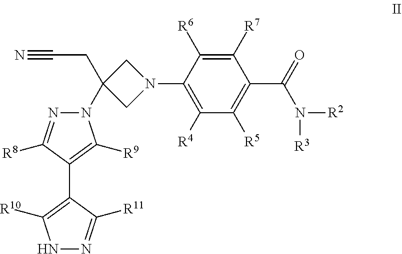

or a pharmaceutically acceptable salt thereof, wherein:
R² is C₁₋₆ alkyl, C₁₋₆ haloalkyl, C₃₋₆ cycloalkyl, or C₃₋₆ cycloalkyl-C₁₋₃ alkyl, wherein said C₁₋₆ alkyl, C₃₋₆ cycloalkyl, and C₃₋₆ cycloalkyl-C₁₋₃ alkyl, are each optionally substituted with 1, 2, or 3 substituents independently selected from fluoro, —CF₃, and methyl;
R³ is H or methyl;
R⁴ is H, F, or Cl;
R⁵ is H or F;
R⁶ is H or F;
R⁷ is H or F;
R⁸ is H or methyl;
R⁹ is H or methyl;
R¹⁰ is H or methyl; and
R¹¹ is H or methyl.

In some embodiments, the JAK1 pathway inhibitor is a compound of Formula III

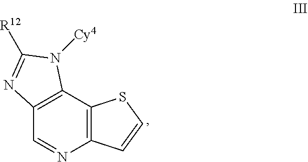

or a pharmaceutically acceptable salt thereof, wherein:
Cy⁴ is a tetrahydro-2H-pyran ring, which is optionally substituted with 1 or 2 groups independently selected from CN, OH, F, Cl, C₁₋₃ alkyl, C₁₋₃ haloalkyl, cyano-C₁₋₃ alkyl, HO—C₁₋₃ alkyl, amino, C₁₋₃ alkylamino, and di(C₁₋₃ alkyl)amino, wherein said C₁₋₃ alkyl and di(C₁₋₃ alkyl)amino is optionally substituted with 1, 2, or 3 substituents independently selected from F, Cl, C₁₋₃ alkylaminosulfonyl, and C₁₋₃ alkylsulfonyl; and
R¹² is —CH₂—OH, —CH(CH₃)—OH, or —CH₂—NHSO₂CH₃.

In some embodiments, the compound of Formula III is ((2R,5S)-5-{2-[(1R)-1-hydroxyethyl]-1H-imidazo[4,5-d]thieno[3,2-b]pyridin-1-yl}tetrahydro-2H-pyran-2-yl)acetonitrile, or a pharmaceutically acceptable salt thereof.

In some embodiments, the JAK1 pathway inhibitor, or a pharmaceutically acceptable salt thereof, is administered in a daily amount of from about 10 mg to about 100 mg on a free base basis. Accordingly, in some embodiments, the selective JAK1 pathway inhibitor is administered in a daily amount of about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg on a free base basis. In some embodiments, the JAK1 pathway inhibitor, or a pharmaceutically acceptable salt thereof, is administered in a daily amount of from about 1 mg to about 100 mg on a free base basis. In some embodiments, the JAK1 pathway inhibitor, or a pharmaceutically acceptable salt thereof, is administered in a daily amount of from about 10 mg to about 80 mg on a free base basis. In some embodiments, the JAK1 pathway inhibitor, or a pharmaceutically acceptable salt thereof, is administered in a daily amount of about 90 mg on a free base basis. In some embodiments, the JAK1 pathway inhibitor, or a pharmaceutically acceptable salt thereof, is administered in a daily amount of about 75 mg on a free base basis. In some embodiments, the JAK1 pathway inhibitor, or a pharmaceutically acceptable salt thereof, is administered in a daily amount of about 45 mg on a free base basis. In some embodiments, the JAK1 pathway inhibitor, or a pharmaceutically acceptable salt thereof, is administered in a daily amount of about 15 mg on a free base basis.

The term “about” means “approximately” (e.g., plus or minus approximately 10% of the indicated value).

In some embodiments, the JAK1 pathway inhibitor, or a pharmaceutically acceptable salt thereof, is administered as one or more sustained release dosage forms each comprising the JAK1 pathway inhibitor, or a pharmaceutically acceptable salt thereof.

In some embodiments, the JAK1 pathway inhibitor, or a pharmaceutically acceptable salt thereof, is administered orally.

The embodiments described herein are intended to be combined in any suitable combination as if the embodiments are multiply dependent claims (e.g., the embodiments related to the selective JAK1 pathway inhibitor and doses of the same, the embodiments related to any salt forms of the compounds disclosed herein, the embodiments related to the individual types of cytokine related diseases or disorders, and the embodiments related to composition and/or administration can be combined in any combination).

All possible combinations are not separately listed herein merely for the sake of brevity.

The compounds described herein can be asymmetric (e.g., having one or more stereocenters). All stereoisomers, such as enantiomers and diastereomers, are intended unless otherwise indicated. Compounds that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

In some embodiments, the compound has the (R)-configuration. In some embodiments, the compound has the (S)-configuration.

Resolution of racemic mixtures of compounds can be carried out by any of numerous methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as 0-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds described herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds described herein can also include isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of a C₁₋₆ alkyl group of Formulae (I), (II), or (III) or a compound of Table 3 can be optionally substituted with deuterium atoms, such as —CD₃ being substituted for —CH₃). The term, “compound,” as used herein is meant to include all stereoisomers, geometric isomers, tautomers, and isotopes of the structures depicted, unless the name indicates a specific stereoisomer. Compounds herein identified by name or structure as one particular tautomeric form are intended to include other tautomeric forms unless otherwise specified.

All compounds, and pharmaceutically acceptable salts thereof, can be found together with other substances such as water and solvents (e.g. hydrates and solvates) or can be isolated.

In some embodiments, the compounds described herein, or salts thereof, are substantially isolated. By “substantially isolated” is meant that the compound is at least partially or substantially separated from the environment in which it was formed or detected. Partial separation can include, for example, a composition enriched in the compounds described herein. Substantial separation can include compositions containing at least about 50%, at least about 60%, at least about 70%, at least about 80%, at least about 90%, at least about 95%, at least about 97%, or at least about 99% by weight of the compounds described herein, or salt thereof. Methods for isolating compounds and their salts are routine in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions, “ambient temperature” and “room temperature” or “rt” as used herein, are understood in the art, and refer generally to a temperature, e.g. a reaction temperature, that is about the temperature of the room in which the reaction is carried out, for example, a temperature from about 20° C. to about 30° C.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “subject”, “individual,” or “patient,” used interchangeably, refers to any animal, including mammals, preferably mice, rats, other rodents, rabbits, dogs, cats, swine, cattle, sheep, horses, or primates, and most preferably humans. In some embodiments, the “subject,” “individual,” or “patient” is in need of said treatment.

In some embodiments, the inhibitors are administered in a therapeutically effective amount. As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

As used herein, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the JAK1 inhibitors can prevent vitiligo in an individual who may be predisposed to the disease. The term “preventing” refers to blocking the occurrence of disease in a patient who may be predisposed to the disease but does not yet experience or display the pathology or symptomatology of the disease.

### Combination Therapies

The methods described herein can further comprise administering one or more additional therapeutic agents. The one or more additional therapeutic agents can be administered to a patient simultaneously or sequentially. The one or more additional therapeutic agents can be administered using different methods than Compound 1 (e.g., topically).

In some embodiments, the additional therapeutic agent is selected from other JAK inhibitors. Additional JAK inhibitors may include ATI-50002 (JAK1/3 selective). Additional JAK inhibitors may include PF-06651600 (JAK3 selective). Additional JAK inhibitors may include PF06700841 (JAK1/TYK2 selective). Additional JAK inhibitors may include Upadacitinib. Additional JAK inhibitors may include Abrocitinib (JAK1 selective). Additional JAK inhibitors may include Cerdulatinib (JAK1/SYK selective). Additional JAK inhibitors may include Deucravacitinib (TYK2 selective).

In some embodiments, the additional therapeutic agent is selected from antioxidants. Antioxidants may be selected from pseudocatalase, vitamin E, vitamin C, ubiquinone, lipoic acid, Polypodium leucotomos, catalase/superoxide dismutase combination, and Ginkgo biloba. In some embodiments, antioxidants may be further administered in combination with phototherapy. The administration of antioxidants during or before phototherapy aims to counteract the oxidative stress induced by UV radiation itself, increasing the phototherapy effectiveness.

In some embodiments, the additional therapeutic agent is a wingless-related integration site (Wnt) agonist. Wnt agonists may include SKL2001.

In some embodiments, the additional therapeutic agent is a melanocortin 1 receptor (MC1R) agonist.

In some embodiments, the additional therapeutic agent is an inflammatory mediator.

Inflammatory mediators may include Prostaglandin E2 (PGE₂) and Bimatoprost (synthetic analog of prostaglandin F2α).

In some embodiments, the additional therapeutic agent is an antimetabolite. Antimetabolites may include 5-fluorouracil.

In some embodiments, the additional therapeutic agent includes Plasmid HSP70i gene therapy. Plasmid HSP70i gene therapy may include HSP70iQ435A.

In some embodiments, the additional therapeutic agent is selected from topical corticosteroids, immunomodulators, calcineurin inhibitors, and phototherapy. In some embodiments, the additional therapies are systemic steroids or immunosuppressants.

In some embodiments, the additional therapeutic agent includes steroids (e.g., orally administered steroids) including systemic steroids. Steroid treatment may include oral steroid minipulse therapy (e.g., using betamethasone and/or dexamethasone).

In some embodiments, topical corticosteroids are selected from augmented betamethasone dipropionate, clobetasol propionate, diflorasone diacetate, halobetasol propionate amcinonide, betamethasone valerate, desoximetasone, diflorasone diacetate, fluocinolone acetonide, halcinonide, and triamcinolone acetonide.

In some embodiments, the additional therapeutic agent includes immunomodulators. Immunomodulators may include PDE4 inhibitors (e.g., apremilast (e.g., orally) or crisaborole (e.g., topically)). Immunomodulators may include anti-CD20 therapy (e.g., ofatumumab). Immunomodulators may include anti-CD19 therapy (e.g., tafasitamab). Immunomodulators may include anti-IL15 therapy (e.g., AMG 714 monoclonal antibody). Immunomodulators may include anti-IL36 therapy (e.g., imsidolimab and spesolimab). Immunomodulators may include anti-TNFalpha therapy (e.g., etanercept and infliximab). Immunomodulators may include anti-CD122 therapy.

In some embodiments, immunomodulators are selected from apremilast, crisaborole, afamelanotide, rituximab, ofatumumab, tafasitamab, minocycline, latanoprost, zinc, tofacitinib, AMG 714 monoclonal antibodies, imsidolimab, spesolimabcyclosporine, etanercept, infliximab, cyclophosphamide, ciclosporin, methotrexate, and sodium oxo-dihydro-acridinylacetate (ODHAA).

In some embodiments, calcineurin inhibitors are selected from tacrolimus (FK-506) and pimecrolimus.

In some embodiments, phototherapy includes exposure to ultraviolet (e.g., excimer lamps or lasers).

In some embodiments, the additional therapeutic agent is a Janus kinase inhibitor. In some embodiments, the Janus kinase inhibitor is ruxolitinib, or a pharmaceutically acceptable salt thereof. In some embodiments, the ruxolitinib is administered topically. The topical administration of ruxolitinib is described in greater detail, for example, in US Patent Publ. No. 2011/0288107, filed Mar. 20, 2011, and Persaud et al., “Plasma pharmacokinetics and distribution of ruxolitinib into skin following oral and topical administration in minipigs” Int J Pharm, 590:119889, Nov. 30, 2020, PMID: 32949620, which are incorporated herein by reference in their entirety.

In some embodiments, the additional therapeutic agent is an IL-6 antagonist or receptor antagonist. In some embodiments, the IL-6 receptor antagonist is tocilizumab.

In some embodiment, the methods described herein can further comprise the use in combination with surgical techniques. In some embodiments, surgical techniques include surgical skin grafts.

### Pharmaceutical Formulations and Dosage Forms

When employed as pharmaceuticals, the JAK1 pathway inhibitors or pharmaceutically acceptable salts thereof, can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, foams, liquids and powders. Conventional pharmaceutical carriers, aqueous, powder or oily bases, thickeners and the like may be necessary or desirable.

This invention also includes pharmaceutical compositions which contain, as the active ingredient, the JAK1 pathway inhibitor described herein, or a pharmaceutically acceptable salt thereof, in combination with one or more pharmaceutically acceptable carriers (excipients). In some embodiments, the composition is suitable for topical administration. In making the compositions, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The JAK1 pathway inhibitors may be milled using known milling procedures such as wet milling to obtain a particle size appropriate for tablet formation and for other formulation types. Finely divided (nanoparticulate) preparations of the JAK1 selective inhibitors can be prepared by processes known in the art, e.g., see International App. No. WO 2002/000196.

The compositions can be formulated in a unit dosage form, each dosage containing a set amount of the active ingredient as the free form or a salt form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

Similar dosages may be used of the compounds described herein in the methods and uses of the invention.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present invention can vary according to, for example, the particular use for which the treatment is made, the manner of administration of the compound, the health and condition of the patient, and the judgment of the prescribing physician. The proportion or concentration of a compound described herein in a pharmaceutical composition can vary depending upon a number of factors including dosage, chemical characteristics (e.g., hydrophobicity), and the route of administration. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the invention can further include one or more additional pharmaceutical agents such as a chemotherapeutic, steroid, anti-inflammatory compound, or immunosuppressant, examples of which are listed herein.

### Kits

The present invention also includes pharmaceutical kits useful, for example, in the treatment and/or prevention of vitiligo, which include one or more containers containing a pharmaceutical composition comprising a therapeutically effective amount of a compound described herein. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. The compounds of the Examples have been found to be JAK inhibitors according to at least one assay described herein.

### Example A: In Vitro JAK Kinase Assay

JAK1 pathway inhibitors that can be used for the treatment of cytokine-related diseases or disorders were tested for inhibitory activity of JAK targets according to the following in vitro assay described in Park et al., Analytical Biochemistry 1999, 269, 94-104. The catalytic domains of human JAK1 (a.a. 837-1142), JAK2 (a.a. 828-1132) and JAK3 (a.a. 781-1124) with an N-terminal His tag were expressed using baculovirus in insect cells and purified. The catalytic activity of JAK1, JAK2 or JAK3 was assayed by measuring the phosphorylation of a biotinylated peptide. The phosphorylated peptide was detected by homogenous time resolved fluorescence (HTRF). IC₅₀s of compounds were measured for each kinase in the 40 microL reactions that contain the enzyme, ATP and 500 nM peptide in 50 mM Tris (pH 7.8) buffer with 100 mM NaCl, 5 mM DTT, and 0.1 mg/mL (0.01%) BSA. For the 1 mM IC₅₀ measurements, ATP concentration in the reactions is 1 mM. Reactions were carried out at room temperature for 1 hour and then stopped with 20 μL 45 mM EDTA, 300 nM SA-APC, 6 nM Eu-Py20 in assay buffer (Perkin Elmer, Boston, MA). Binding to the Europium labeled antibody took place for 40 minutes and HTRF signal was measured on a Fusion plate reader (Perkin Elmer, Boston, MA). The compounds in Table 3 were tested in this assay and shown to have the IC₅₀ values also found in Table 3.

### Example B: In Vitro JAK Kinase Assay Using Melanocyte Proliferation Assay with 20% CD8+ T Cell Conditioned Media

The assay investigates an in vitro system using CD8+ T Cells and melanocytes. The assay tests whether Compound 1 modulates the inflammatory response and how such modulation affects melanocyte biology.

Methods: CD8+ T cell Conditioned Media Step: Normal Human CD8+ T Cells from peripheral blood were purchased (STEMCELL Technologies). They were cultured for 48 hours at 37 C and 5% CO₂ under activating and unactivating conditions inside the Incucyte Live-Cell Analysis System (Essen Bioscience). Activated T cells were cultured in a flat bottom 96 well plate (Costar) that was first coated with 10 ug/ml anti-CD3 (BD Biosciences) for 2 hours at room temperature and then washed one time with PBS before cells were added. They were grown in Melanocyte Growth Medium (NHM-GM, MatTek) containing 1 ug/ml anti-CD28 (BD Biosciences). Unactivated T cells were treated the same way except that they were cultured in a separate plate without anti-CD3 and anti-CD28. Each T cell plate was treated at seeding with Compound 1 (1000, 100, or 10 nM) or media alone. Each treatment was done in triplicate. After 48 hours, all the samples (T cells and media) were transferred to new V-bottom plates and centrifuged at 1300 RPMs for 10 minutes. Supernatants were collected from the T cell pellets carefully to not disturb the pellet. The supernatants (CD8+ T cell Conditioned Media) used fresh to treat the melanocyte. The remaining amounts were stored at −80 C and then thawed for protein analysis by Luminex technology (custom Procarta 45-plex, Thermo Fisher Scientific). The treated CD8+ T cell pellets were lysed using the Quantigene sample processing kit reagents following the kit instructions. Lysates were stored at −80 C and then thawed for RNA analysis by Luminex technology using two custom Quantigene 80-Plexes—150 targeted genes total (Thermo Fisher Scientific).

Melanocyte Proliferation Step: Normal Human Melanocytes isolated from a black donor's neonatal foreskin epidermis were purchased (MatTek). They were thawed and seeded following manufacturer's instruction in NHM-GM media in flat bottom 96 well plates (Costar) for 18 hours at 37 C and 5% CO₂. They were approximately 10% confluent when treated with 20% CD8+ T cell Conditioned Media (25 ul Conditioned media added to 100 ul of NHM-GM already in the wells). They were cultured for 48 hours at 37 C and 5% CO₂ inside the Incucyte Live-Cell Analysis System (Essen Bioscience) with 5 images per well taken every 3 hrs. After 48 hours, all the supernatants were transferred from the melanocyte plates to new V-bottom plates and centrifuged at 1300 RPMs for 10 minutes. Supernatants were collected and were stored at −80 C and then thawed for protein analysis by Luminex technology (custom Procarta 45-plex, Thermo Fisher Scientific). The treated melanocytes were lysed using the Quantigene sample processing kit reagents following the kit instructions. Lysates were stored at −80 C and then thawed for RNA analysis by Luminex technology using two custom Quantigene 80-Plexes for a total of 150 targeted genes (Thermo Fisher Scientific). Incucyte images were analyzed using the Incucyte software and the percentage of melanocyte confluence was determined for each treatment.

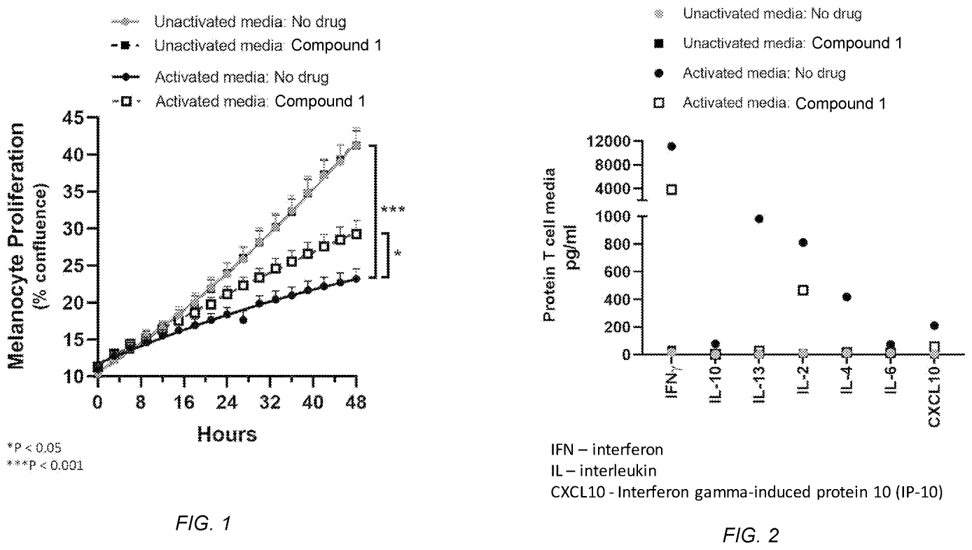

Results: FIG. 1 is a graph depicting melanocyte proliferation, % confluence vs. time (hours). Compound 1 does not directly alter melanocyte proliferation following transfer of 20% unactivated T cell media. Addition of 20% media from CD3/CD28-activated human CD8+ T cells to the NHM-GM in vitro culture significantly inhibited melanocyte proliferation compared to unactivated conditions. Compound 1 (1000 nM) co-cultured with the CD3/CD28-activated human CD8+ T cells partially reversed the suppressed melanocyte proliferation response. This improvement of melanocyte proliferation was statistically significant. Compound 1 added to the inflammatory culture media results in a significant reduction of spontaneous proliferation compared to thethe natural melanocyte proliferation rate. It is a time-dependent reduction of the spontaneous natural proliferation response.

FIG. 2 is a graph depicting the effect of Compound 1 on JAK1-dependent cytokines using T-cell compartments, protein T cell media (pg/ml) vs. specific JAK1-dependent cytokines. Consistent with previously reported data Compound 1 co-cultured with the CD3/CD28-activated human CD8+ T cells inhibits inflammatory cytokines that are JAK-STAT dependent.

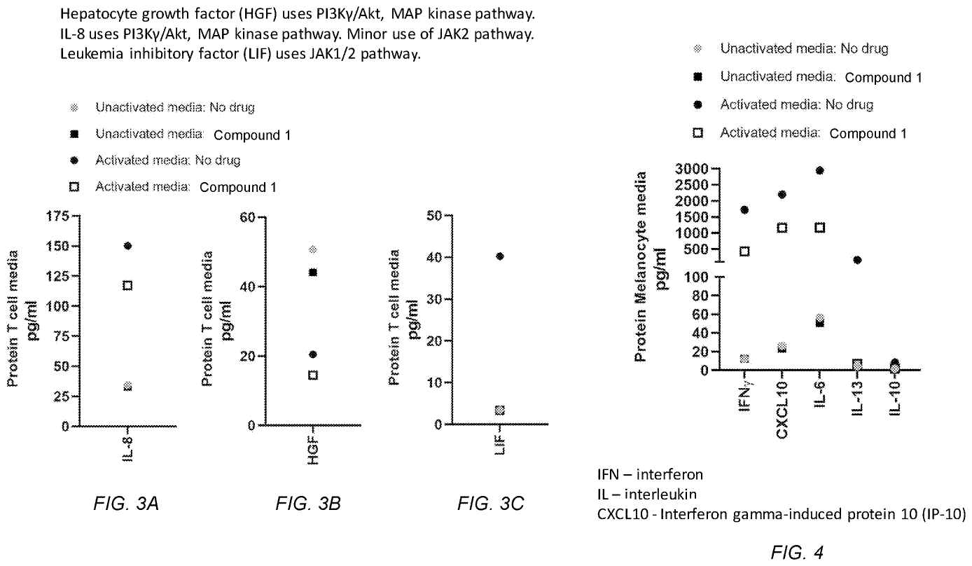

FIGS. 3A-C are graphs depicting the effect of Compound 1 on growth factors and chemokine using T-cell compartments, protein T cell media (pg/ml) vs. IL-8 (A), HGF (B), and LIF (C). Compound 1 co-cultured with the CD3/CD28-activated human CD8+ T cells reduced several additional mediators that are not obligated to use JAK1 signaling.

FIG. 4 is a graph depicting the effect of Compound 1 on JAK1-dependent cytokines using melanocytes, protein melanocyte media (pg/ml) vs. specific JAK1-dependent cytokines. Compound 1 reduces inflammatory cytokines that are JAK-STAT dependent. Several of these cytokines are upregulated by melanocytes under stress conditions (IFNγ, CXCL10, IL-6).

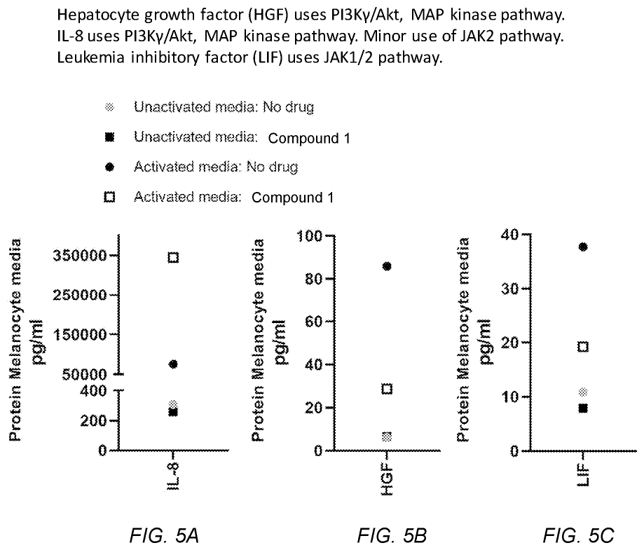

FIGS. 5A-C are graphs depicting the effect of Compound 1 on growth factors and chemokine using melanocytes, protein melanocyte media (pg/ml) vs. IL-8 (A), HGF (B), and LIF (C). IL-8 is upregulated by melanocytes under stress conditions and this is increased in Compound 1 conditions.

Discussion: Hepatocyte Growth Factor (HGF)/mesenchymal-epithelial transition factor (c-MET) signaling protects melanocytes from apoptosis and stimulates their proliferation and motility. HCF concentrations increased in melanocyte culture supernatants following transfer of activated T cell conditioned media. Compound 1 significantly reduced HGF levels (~67%) but melanocyte proliferation was increased. Based on the data (melanocytes and T cells), reduction of HGF expression by Compound 1, via a signaling mechanism, would be expected to be detrimental on melanocyte proliferation.

Interleukin-8 (IL-8/CXCL8) is reported to potentiate the proliferative response of melanocytes to multiple growth factors via binding to its cognate receptors (CXCR1 & CXCR2). IL-8 production and CXCR1/2 signaling are both JAK1 independent. Compound 1 treatment of CD8+ T cells slightly reduced IL-8 concentration (↓22%) however there was a profound increase (↑~450%) in the melanocyte culture. Based on the data (melanocytes and T cells), unexpected upregulation of IL-8 levels in melanocyte cultures was observed that would enhance proliferative response.

Leukemia inhibitory factor (LIF) increases primary melanocyte proliferation and differentiation. LIF receptor signaling is JAK1-dependent and therefore Compound 1 would be expected to have a detrimental affect on melanocyte proliferation. Unexpectedly, melanocyte proliferation is increased. Based on the data (T cells), on-target inhibition of LIF is not matched by enhanced melanocyte proliferation response.

Compound 1 can downregulate inflammatory cytokines in a CD8+ activated T cell microenvironment. It allows melanocyte proliferation to normalize and also stops the generation of inflammatory mediators from the melanocyte.

### Example C: Phase 2 Study of Compound 1

### Study Design

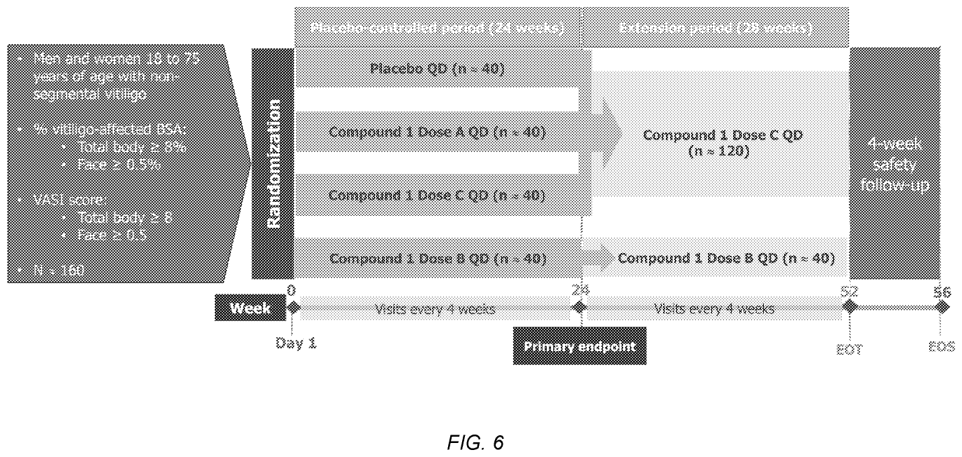

FIG. 6 depicts an outline of a phase 2 randomized, double-blind, placebo-controlled dose-ranging study of the efficacy and safety of Compound 1. A study is conducted using Compound 1 for treating participants with non-segmental vitiligo. The study can include a 28-week double-blind extension period in participants with vitiligo. Participants can include those with non-segmental vitiligo who have depigmented areas ≥8% total BSA, ≥8 T-VASI, ≥0.5% BSA on the face, and >0.5 F-VASI. Participants will be stratified based on total BSA involvement (8-20% and >20%). In some embodiments, participants can include those with non-segmental vitiligo who have depigmented areas ≥5% total BSA including ≥5 T-VASI, ≥0.5% BSA on the face and ≥0.5 F-VASI. Participants will be stratified based on total BSA involvement (8-20% and >20%). The study can include men and women aged 18 to 75 years of age.

The 24-week placebo-controlled double-blind period will randomize approximately 160 participants 1:1:1:1 to 1 of 3 treatment groups (Dose A, Dose B, or Dose C) or the placebo group. The 28-week double-blind extension will include all participants who successfully complete the placebo-controlled period. In some embodiments, the JAK1 pathway inhibitor, or a pharmaceutically acceptable salt thereof, will be administered in a daily amount of about Dose B on a free base basis. In some embodiments, the JAK1 pathway inhibitor, or a pharmaceutically acceptable salt thereof, will be administered in a daily amount of about Dose C on a free base basis. The primary endpoint is the Percent change from baseline in Total Vitiligo Area Scoring Index (T-VASI) at Week 24, and the key secondary endpoint is the proportion of participants achieving T-VASI50 at Week 24. In some embodiments, the outcome of this study will be instrumental in informing dose selection to be evaluated in a Phase 3 study.

In some embodiments, criteria for participants to be excluded from the study include any one of the following: other skin depigmentation disorders (e.g., piebaldism, pityriasis alba, leprosy, postinflammatory hypopigmentation, progressive macule hypomelanosis, nevus anemicus, chemical leukoderma, and tinea versicolor); uncontrolled thyroid function at screening as determined by the investigator (Note: If the participant has a history of thyroid disease and is on treatment, the participant must be on a stable thyroid regimen for at least three months prior to Day 1); use of laser or light-based treatment (phototherapy), including tanning beds within 8 weeks prior to Day 1; use of dihydroxyacetone (generally present in self-tanning products) within 4 weeks prior to Day 1; current or past use of the depigmenting agent monobenzyl ether of hydroquinone, including Benoquin® (monobenzone); history of melanocyte-keratinocyte transplantation procedure (MKTP) or other surgical treatment for vitiligo; spontaneous and significant repigmentation within 6 months prior to screening (eg, repigmentation without any treatment, and significant in amount as determined by the investigator); women who are pregnant or who are considering pregnancy, or breastfeeding; history of treatment failure to vitiligo, or any other inflammatory condition, with any systemic or topical JAK or TYK2 inhibitor (e.g., ruxolitinib, tofacitinib, baricitinib, filgotinib, lestaurtinib, pacritinib, abrocitinib, brepocitinib); current use of anticoagulants or medications known to cause thrombocytopenia; hepatitis: evidence of HBV or HCV infection or risk of reactivation (participant cannot be positive for HBV DNA, HCV RNA, HBsAg, or anti-hepatitis B core antibody; however, participants with no prior history of HBV infection who have been vaccinated against HBV and who have a positive antibody against HBV surface antigen (anti-HBs+) as the only evidence of prior exposure may participate in the study); known HIV infection; known hypersensitivity or severe reaction to Compound 1 or excipients of Compound 1; inability or unlikeliness of the participant to comply with the dose schedule and study evaluations, in the opinion of the investigator; and any condition that would, in the investigator's judgment, interfere with full participation in the study, including administration of study drug and attending required study visits; pose a significant risk to the participant; or interfere with interpretation of study data.

In some embodiments, criteria for participants to be excluded from the study include participants with concurrent conditions or history of other diseases, including any one of the following: thrombocytopenia, coagulopathy, platelet dysfunction, or history of thrombotic events; any clinically significant medical condition other than vitiligo, as determined by the investigator, that is not adequately controlled with appropriate treatment or may interfere with the course, severity, or assessments of this study; any other active skin disease or condition that may interfere with the course, severity, or assessments of this study; any bacterial, fungal, or viral infection, based on the investigator's clinical assessment, make the participant an unsuitable candidate for the study; current herpes zoster infection, a history of disseminated herpes simplex, or a history of herpes zoster; history of malignancy, including melanoma, lymphoma and leukemia within 5 years before Day 1, other than a successfully treated nonmetastatic cutaneous squamous cell carcinoma, basal cell carcinoma, or localized carcinoma in situ of the cervix; and albinism.

In some embodiments, criteria for participants to be excluded from the study include receipt of medications or investigational drugs within the following interval before Day 1 (first administration of study drug): <12 weeks or 5 half-lives (if known), whichever is longer, for any topical or systemic JAK or TYK2 inhibitor; <12 weeks or 5 half-lives (if known), whichever is longer, for any investigational or experimental treatments; <12 weeks or 5 half-lives (if known), whichever is longer, for systemic immunosuppressive or immunomodulating biologic drugs (eg, egadalimumab, etanercept, infliximab, golimumab, certolizumab, ustekinumab, secukinumab, brodalumab, ixekizumab, risankizumab, guselkumab, bimekizumab, iscalimab, bermekimab, rituximab, anakinra); <6 weeks for live vaccine, or planning to receive live vaccine during the course of the study or within 6 weeks after the last dose of study drug; <4 weeks for systemic immunosuppressive or immunomodulating small molecule drugs (e.g., oral or injectable corticosteroids, methotrexate, cyclosporine, dapsone, azathioprine); <3 weeks for any oral or topical PDE-4 inhibitor (eg, apremilast, crisaborole); <2 weeks for any topical drug applied onto vitiligo lesions; <2 weeks for any OTC therapies used for vitiligo treatment; <2 weeks or 5 half-lives (if known), whichever is longer, for strong and moderate systemic CYP3A4 inhibitors and strong systemic CYP3A4 inducers within 2 weeks or 5 half-lives (if known). Examples include but are not limited to the following medications: erythromycin, rifampicin/rifampin, ciprofloxacin, some azole antifungals (eg, ketoconazole, fluconazole), nefazodone, St. John's Wort, diltiazem, mibefradil, verapamil, grapefruit/grapefruit juice, and Seville oranges; and <1 week for anti-platelet.

In some embodiments, criteria for participants to be excluded from the study include use of systemic immunosuppressive or immunomodulating small molecule drugs note: that use of corticosteroid inhalers and intranasal sprays is allowed; use of oral corticosteroids for nondermatologic conditions (e.g., asthma exacerbation, bronchitis) is allowed for no longer than 7 days, if deemed acceptable by the investigator and the sponsor; and use of topical corticosteroids for dermatologic disease besides vitiligo (e.g., atopic dermatitis or psoriasis) is allowed for areas not being treated for vitiligo (the total BSA involvement for other dermatologic diseases, outside of the areas treated for vitiligo, must not exceed 10%).

In some embodiments, criteria for participants to be excluded from the study include use of systemic immunosuppressive or immunomodulating small molecule drugs note low dose acetyl salicylic acid (≤100 mg QD) is permitted for the purpose of cardiovascular prophylaxis at the discretion of the investigator.

In some embodiments, criteria for participants to be excluded from the study include evidence of active or latent or inadequately treated infection with Mycobacterium tuberculosis (i.e., TB) as defined by at least one of the following: a positive QuantiFERON®-TB Gold In-Tube test (QFT-GIT) or positive Mantoux/PPD tuberculin skin test performed at or within the 12 weeks prior to Day 1 is exclusionary and a negative test is required for eligibility, unless other criteria as described here are applicable (note it is recommended that participants with a history of Bacille Calmette Guerin vaccination be tested with the QFT-GIT, since the Mantoux/PPD tuberculin skin test may be positive due to vaccination (note a QFT-GIT or Mantoux/PPD tuberculin skin test is not required if the participant has previously received a documented adequate course of therapy for either latent or active TB infection)); a history of either untreated or inadequately treated latent or active TB infection; if a participant has previously received an adequate course of therapy for either latent (9 months of isoniazid in a locale where rates of primary multidrug TB resistance are <5% or an acceptable alternative regimen) or active (acceptable multidrug regimen) TB infection, neither a QFT-GIT nor a Mantoux/PPD tuberculin skin test is needed, but a chest radiograph(s) or other appropriate diagnostic image, performed within 3 months of Day 1, is required (note to be considered eligible for the study, the radiograph(s) must be negative for active tuberculosis infection as determined by a qualified radiologist and documentation of adequate treatment for TB and negative chest radiograph(s) results must be obtained prior to Day 1; and a participant who is currently being treated for active TB infection.

In some embodiments, criteria for participants to be excluded from the study include participants with laboratory values at screening defined in Table 4:

TABLE 4

| | |
|---|---|
| Laboratory Parameter | Exclusion Criterion |

Hematology

| | | |
|---|---|---|
| a | Platelets | <150 × 10⁹/L |
| b | Hemoglobin | <10 g/L |
| c | ANC | <1.5 × 10⁹/L |
| d | WBCs | ≤3.0 × 10⁹/L |

Hepatic

| | | |
|---|---|---|
| e | ALT | ≥2 × ULN |
| f | AST | ≥2 × ULN |
| g | Total bilirubin | ≥1.5 × ULN |
| | | (Note: unless clinical diagnosis |
| | | of Gilbert's syndrome) |
| h | Alkaline phosphatase | ≥2 × ULN |

Renal

| | | |
|---|---|---|
| j | Serum creatinine | >1.25 × ULN |

Coagulation

| | | |
|---|---|---|
| l | PT (and calculated INR) | >ULN |
| m | INR | >ULN |

### Example D: In Vitro JAK Kinase Assay Using Melanocyte Proliferation Assay with 20% CD8+ T Cell Conditioned Media

The assay investigates an in vitro system using CD8+ T Cells and melanocytes. The assay tests whether ruxolitinib modulates the inflammatory response and how such modulation affects melanocyte biology.

Methods: CD8+ T cell Conditioned Media Step: Normal Human CD8+ T Cells from peripheral blood were purchased (STEMCELL Technologies). They were cultured for 48 hours at 37 C and 500 CO₂ under activating and unactivating conditions inside the Incucyte Live-Cell Analysis System (Essen Bioscience). Activated T cells were cultured in a flat bottom 96 well plate (Costar) that was first coated with 10 ug/ml anti-CD3 (BD Biosciences) for 2 hours at room temperature and then washed one time with PBS before cells were added. They were grown in Melanocyte Growth Medium (NHM-GM, MatTek) containing 1 ug/ml anti-CD28 (BD Biosciences). Unactivated T cells were treated the same way except that they were cultured in a separate plate without anti-CD3 and anti-CD28. Each T cell plate was treated at seeding with ruxolitinib (1000, 100, or 10 nM) or media alone. Each treatment was done in triplicate. After 48 hours, all the samples (T cells and media) were transferred to new V-bottom plates and centrifuged at 1300 RPMs for 10 minutes. Supernatants were collected from the T cell pellets carefully to not disturb the pellet. The supernatants (CD8+ T cell Conditioned Media) used fresh to treat the melanocyte. The remaining amounts were stored at −80 C and then thawed for protein analysis by Luminex technology (custom Procarta 45-plex, Thermo Fisher Scientific). The treated CD8+ T cell pellets were lysed using the Quantigene sample processing kit reagents following the kit instructions. Lysates were stored at −80 C and then thawed for RNA analysis by Luminex technology using two custom Quantigene 80-Plexes—150 targeted genes total (Thermo Fisher Scientific).

Melanocyte Proliferation Step: Normal Human Melanocytes isolated from a black donor's neonatal foreskin epidermis were purchased (MatTek). They were thawed and seeded following manufacturer's instruction in NHM-GM media in flat bottom 96 well plates (Costar) for 18 hours at 37 C and 5% CO₂. They were approximately 10% confluent when treated with 20% CD8+ T cell Conditioned Media (25 ul Conditioned media added to 100 ul of NHM-GM already in the wells). They were cultured for 48 hours at 37 C and 5% CO₂ inside the Incucyte Live-Cell Analysis System (Essen Bioscience) with 5 images per well taken every 3 hrs. After 48 hours, all the supernatants were transferred from the melanocyte plates to new V-bottom plates and centrifuged at 1300 RPMs for 10 minutes. Supernatants were collected and were stored at −80 C and then thawed for protein analysis by Luminex technology (custom Procarta 45-plex, Thermo Fisher Scientific). The treated melanocytes were lysed using the Quantigene sample processing kit reagents following the kit instructions. Lysates were stored at −80 C and then thawed for RNA analysis by Luminex technology using two custom Quantigene 80-Plexes for a total of 150 targeted genes (Thermo Fisher Scientific). Incucyte images were analyzed using the Incucyte software and the percentage of melanocyte confluence was determined for each treatment.

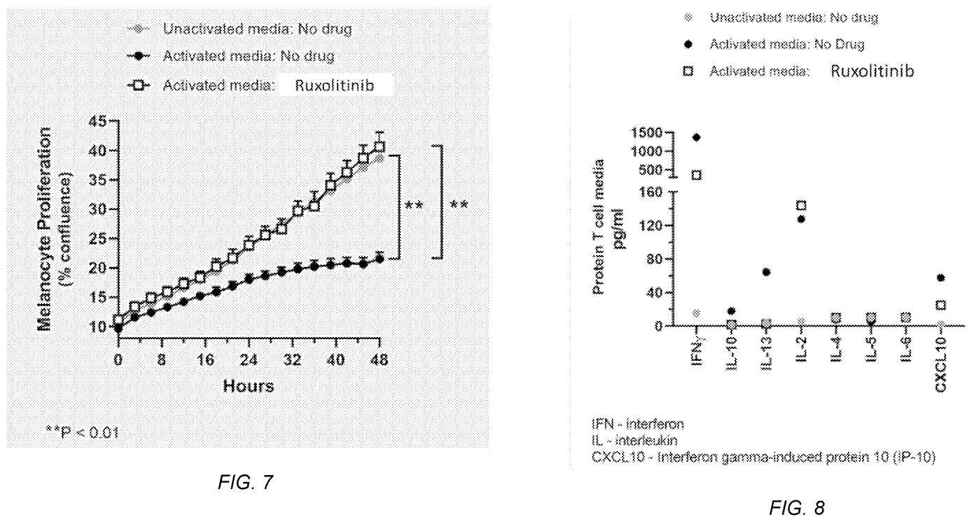

Results: FIG. 7 is a graph depicting melanocyte proliferation, % confluence vs. time (hours). Addition of 20% media from CD3/CD28-activated human CD8+ T cells to the NHM-GM in vitro culture significantly inhibited melanocyte proliferation compared to unactivated conditions. Ruxolitinib (1000 nM) co-cultured with the CD3/CD28-activated human CD8+ T cells appeared to completely reverse the suppressed melanocyte proliferation response. This improvement of melanocyte proliferation was statistically significant. Ruxolitinib added to the inflammatory culture media results in a significant reduction of spontaneous proliferation compared to the natural melanocyte proliferation rate. It is a time-dependent reduction of the spontaneous natural proliferation response.

FIG. 8 is a graph depicting the effect of ruxolitinib on JAK1-dependent cytokines using T-cell compartments, protein T cell media (pg/ml) vs. specific JAK1-dependent cytokines. Consistent with previously reported data ruxolitinib co-cultured with the CD3/CD28-activated human CD8+ T cells inhibits inflammatory cytokines that are JAK-STAT dependent except for inflammatory cytokine IL-2 which is upregulated (there appears to be no effect for inflammatory cytokines IL-4, IL-5 (it is noted that IL-5 is JAK2/2), and IL-6).

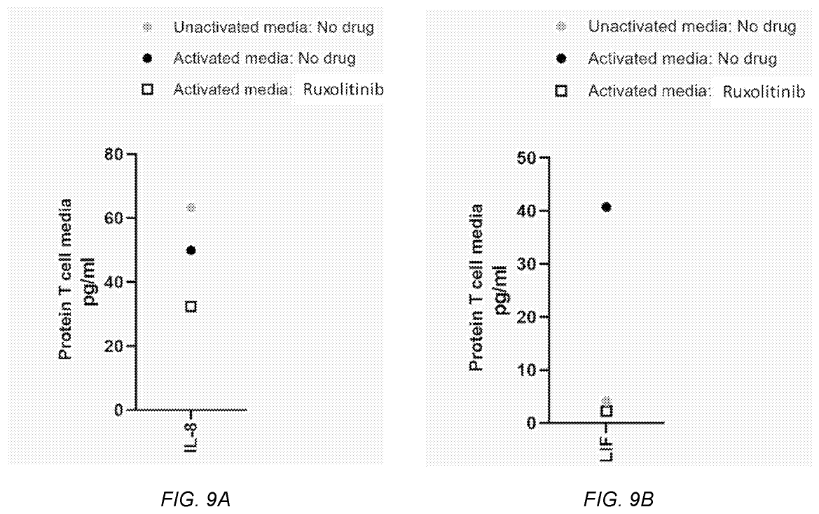

FIGS. 9A-B are graphs depicting the effect of ruxolitinib on growth factors and chemokine using T-cell compartments, protein T cell media (pg/ml) vs. IL-8 (A) and LIF (B). Ruxolitinib co-cultured with the CD3/CD28-activated human CD8+ T cells reduced several additional mediators that are not obligated to use JAK1 signaling.

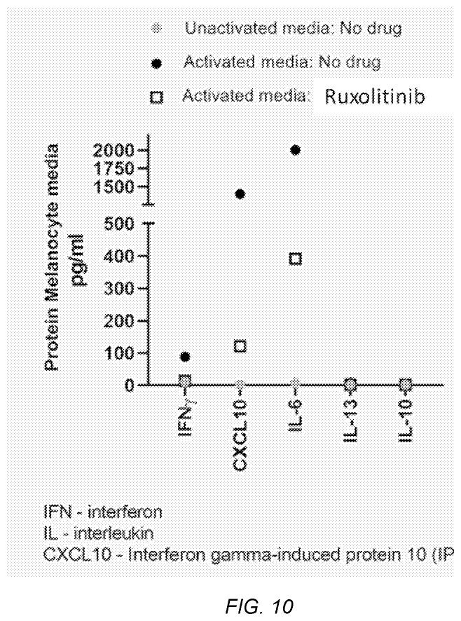

FIG. 10 is a graph depicting the effect of ruxolitinib on JAK1-dependent cytokines using melanocytes, protein melanocyte media (pg/ml) vs. specific JAK1-dependent cytokines. Ruxolitinib reduces inflammatory cytokines that are JAK-STAT dependent (there appears to be no effect for inflammatory cytokines IL-13 and IL-10). Several of these cytokines are upregulated by melanocytes under stress conditions (IFNγ, CXCL10, IL-6).

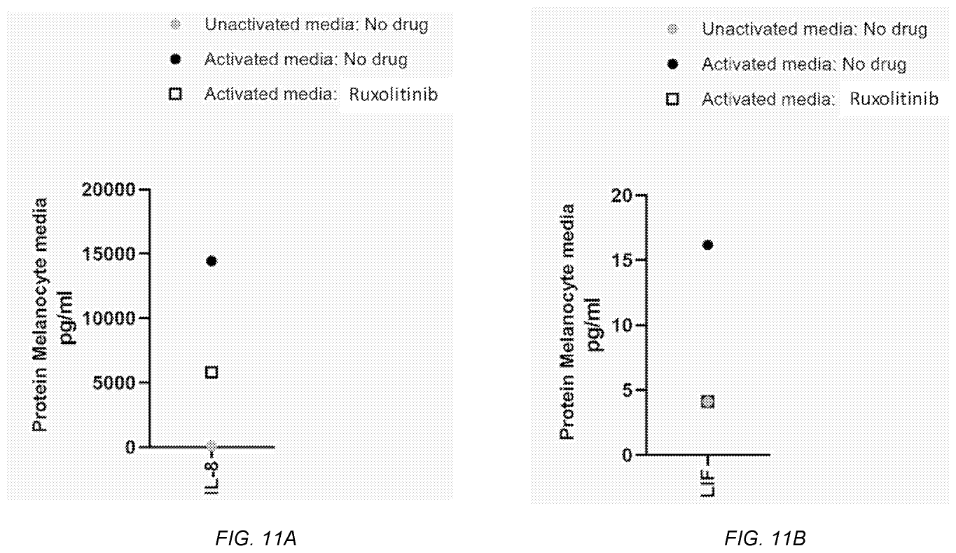

FIGS. 11A-B are graphs depicting the effect of ruxolitinib on growth factors and chemokine using melanocytes, protein melanocyte media (pg/ml) vs. IL-8 (A) and LIF (B). IL-8 is upregulated by melanocytes under stress conditions and this is reduced in ruxolitinib conditions.

Discussion: Interleukin-8 (IL-8/CXCL8) is reported to potentiate the proliferative response of melanocytes to multiple growth factors via binding to its cognate receptors (CXCR1 & CXCR2). IL-8 production and CXCR1/2 signaling are both JAK1 independent. Ruxolitinib treatment of CD8+ T cells slightly reduced IL-8 concentration.

Leukemia inhibitory factor (LIF) increases primary melanocyte proliferation and differentiation. LIF receptor signaling is JAK1-dependent and therefore ruxolitinib would be expected to have a detrimental effect on melanocyte proliferation. Unexpectedly, melanocyte proliferation is increased. Based on the data (T cells), on-target inhibition of LIF is not matched by enhanced melanocyte proliferation response.

Ruxolitinib can downregulate inflammatory cytokines in a CD8+ activated T cell microenvironment. It allows melanocyte proliferation to normalize and also stops the generation of inflammatory mediators from the melanocyte.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1.-11. (canceled)

12. A method of administering a JAK1 pathway inhibitor in a subject, wherein the method comprises administering to the subject a therapeutically effective amount of JAK1 pathway inhibitor 4-[3-(cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide, or a pharmaceutically acceptable salt thereof, wherein the JAK1 pathway inhibitor, or pharmaceutically acceptable salt thereof, is administered in a daily dose of about 45 mg or about 75 mg on a free base basis.

13. The method of claim 12, wherein the JAK1 pathway inhibitor, or pharmaceutically acceptable salt thereof, is administered in a daily dose of about 45 mg on a free base basis.

14. The method of claim 12, wherein the JAK1 pathway inhibitor, or pharmaceutically acceptable salt thereof, is administered in a daily dose of about 75 mg on a free base basis.

15. The method of claim 12, wherein the JAK1 pathway inhibitor is a pharmaceutically acceptable salt of 4-[3-(cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide.

16. The method of claim 12, wherein the JAK1 pathway inhibitor is 4-[3-(cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide phosphoric acid salt.

17. The method of claim 12, wherein the JAK1 pathway inhibitor, or pharmaceutically acceptable salt thereof, is administered in combination with a further therapeutic agent.

18. The method of claim 12, wherein the administering comprises administering the JAK1 pathway inhibitor, or pharmaceutically acceptable salt thereof, together with at least one pharmaceutically acceptable carrier.

19. The method of claim 12, wherein the subject is human.

20. The method of claim 12, wherein the method comprises treating a dermatological disease.

21. The method of claim 12, wherein the method comprises treating a chronic skin disorder.

22. The method of claim 12, wherein the method comprises treating an inflammatory condition.
